(19) United States
(12) Patent Application Publication
(10) Pub. No.: US 2021/0261423 A1
(43) Pub. Date: Aug. 26, 2021

(54) AEI-TYPE ZEOLITIC MATERIAL OBTAINED FROM HIGH TEMPERATURE CALCINATION AND USE AS A CATALYST

(71) Applicant: BASF SE, Ludwigshafen am Rhein (DE)

(72) Inventors: Andrei-Nicolae PARVULESCU, Ludwigshafen (DE); Robert MCGUIRE, Florham Park, NJ (US); Ulrich MUELLER, Ludwigshafen (DE); Toshiyuki YOKOI, Midori-ku (JP); Hermann GIES, Bochum (DE); Bernd MARLER, Bochum (DE); Dirk DEVOS, Leuven (BE); Ute KOLB, Mainz (DE); Feng-Shou XIAO, Hangzhou (CN); Weiping ZHANG, Dalian City (CN); Xiangju MENG, Hangzhou (CN); Yong WANG, Midori-ku (JP)

(73) Assignee: BASF SE, Ludwigshafen am Rhein (DE)

(21) Appl. No.: 17/254,050

(22) PCT Filed: Jun. 18, 2019
(86) PCT No.: PCT/CN2019/091741

§ 371 Date: Dec. 18, 2020

(30) Foreign Application Priority Data
Jun. 20, 2018 (CN) PCT/CN2018/091925

(51) Int. Cl. C01B 39/48; C01B 39/02; B01J 29/70; B01J 35/00; C07C 1/20; B01J 6/00; B01J 37/30

(52) U.S. Cl. CPC C01B 39/48; C01B 39/026; B01J 29/70; B01J 35/002; C07C 2529/70; B01J 6/001; B01J 37/30; B01J 2229/40; C07C 1/20

(57) ABSTRACT

A process for preparing a zeolitic material having an AEI-type framework structure having SiO₂ and X₂O₃ in its framework, X standing for a trivalent element, may involve: (1) preparing a mixture of structure directing agent(s) and a first zeolitic material with SiO₂ and X₂O₃ in its framework, the first zeolitic material having a FER, TON, MTT, FAU, GIS, MOR, BEA, MFI, and LTA framework; (2) heating the mixture to obtain a second zeolitic material having an AEI-type framework with SiO₂ and X₂O₃ in its framework; (3) optionally calcining the second zeolitic material; (4) optionally subjecting the zeolitic material from (2) or (3) to ion-exchange, preferably ion-exchanging ionic extra-framework element(s) in the zeolite framework for H⁺ and/or NH₄⁺; (5) calcining the zeolitic material from (2), (3), or (4) at greater than 600 to 900° C., the calcining atmosphere containing less than 10 vol.-% of H₂O. Such zeolites can convert oxygenates to olefins.

### TECHNICAL FIELD

The present invention relates to a process for the preparation of a zeolitic material having an AEI-type framework structure as well as to a zeolitic material having an AEI-type framework structure as such and as obtainable according to the inventive process. Furthermore, the present invention relates to a process for the conversion of oxygenates to olefins using a zeolitic material having an AEI-type framework structure according to the present invention. Finally, the present invention relates to the use of a zeolitic material having an AEI-type framework structure according to the present invention, in particular as a catalyst.

### INTRODUCTION

Zeolitic materials having framework type AEI are known to be potentially effective as catalysts or catalyst components for treating combustion exhaust gas in industrial applications, for example for converting nitrogen oxides (NOₓ) in an exhaust gas stream. Moliner, M. et al. in Chem. Commun. 2012, 48, pages 8264-8266 concerns Cu-SSZ-39 and its use for the SCR of nitrogen oxides NOx, wherein the SSZ-39 is produced with the use of N,N-dimethyl-3,5-dimethylpiperidinium cations as the organotemplate. Maruo, T. et al. in Chem. Lett. 2014, 43, page 302-304 relates to the synthesis of AEI zeolites by hydrothermal conversion of FAU zeolites in the presence of tetraethylphosphonium cations. Martin, N. et al. in Chem. Commun. 2015, 51, 11030-11033 concerns the synthesis of Cu-SSZ-39 and its use as a catalyst in the SCR of nitrogen oxides NOx. As regards the methods of synthesis of the SSZ-39 zeolite in said document, these include the use of N,N-dimethyl-3,5-dimethylpiperidinium cations as well as of tetraethylphosphonium cations. Unpublished international patent application PCT/CN2016/115938 relates to a process for the production of zeolitic materials including materials having the AEI-type framework structure such as SSZ-39. Unpublished international patent application PCT/CN2017/112343 concerns a process for preparing a zeolitic material having an AEI framework structure using a quaternary phosphonium cation.

Zeolitic materials are however highly versatile and known to find broad applications, in particular in catalytic applications.

In view of the decreasing amount of oil reserves which constitute the raw material for the production of short-chain hydrocarbons and derivatives thereof, alternative processes for the production of such base chemicals are of a growing importance. In such alternative processes for the production of short-chain hydrocarbons and derivatives thereof, often highly specific catalysts are used therein for converting other raw materials and/or chemicals to hydrocarbons and their derivatives such as in particular short-chain olefins. A particular challenge involved in such processes not only relies in the optimal choice of reaction parameters but, more importantly, in the use of particular catalysts allowing for the highly efficient and selective conversion to a desired hydrocarbon or derivative thereof such as in particular olefinic fractions. In this respect, processes in which methanol is employed as the starting material, are of particular importance, wherein their catalytic conversion usually leads to a mixture of hydrocarbons and derivatives thereof, in particular olefins, paraffins, and aromatics.

Thus, the particular challenge in such catalytic conversions resides in the optimization and the fine tuning of the catalysts (particularly the zeolite pore structure, acid type and strength) employed as well as the process architecture and parameters such that a high selectivity towards as few products as possible may be achieved. For this reason, such processes are often named after the products for which a particularly high selectivity may be achieved in the process. Accordingly, processes which have been developed in the past decades towards the conversion of oxygenates to olefins and in particular of methanol to olefins which have gained increasing importance in view of dwindling oil reserves are accordingly designated as methanol-to-olefin-processes (MTO-processes for methanol to olefins).

Among the catalytic materials which have been found for use in such conversions, zeolitic materials have proven of high efficiency, wherein in particular zeolitic materials of the pentasil-type and more specifically those having an MFI- and MEL-type framework structures including such zeolites displaying an MFI-MEL-intergrowth type framework structure are employed. On the other hand, U.S. Pat. No. 5,958,370 which relates to the production of SSZ-39 having the AEI type framework structure also describes their use in the catalytic conversion of methanol to olefins.

There however remains a need for providing new materials which may be employed for such conversions, and in particular new materials which display improvements with regard to the catalytic activity and selectivity towards desired products in such reactions, as well as maintaining a high conversion rate and high activities and selectivities over prolonged reaction times and long times on stream in continuous processes.

Although a multitude of zeolitic materials have been produced for a variety of applications, there remains a need not only for finding new applications for known zeolitic materials, but furthermore and more importantly to provide improved methods for their synthesis and production which lead to new and improved properties, in particular in the field of catalysis. More specifically, there remains a need for new and improved methodologies which allow for variations and fine tuning of the zeolitic materials in order to optimize their use depending on the type of catalytic conversion and the specific products or spectrum of products which are desired.

### DETAILED DESCRIPTION

It was therefore an object of the present invention to provide an improved process for preparing a zeolitic material having an AEI-type framework structure which leads to a material displaying improved catalytic properties, in particular in a process for the conversion of oxygenates to olefins such as in the conversion of methanol to olefins. Thus, it has quite unexpectedly been found that the zeolitic materials having an AEI-type framework structure obtained according to the inventive method display specific quantities of acid sites and in particular ratios of the amount of different acid sites to one another. In particular, it has surprisingly been found that the inventive zeolitic materials displaying an AEI-type framework structure display both a considerably improved activity and a surprisingly high selectivity in the conversion of oxygenates to olefins, and in particular of methanol towards C2 to C4 olefins, and in particular towards C3 olefins.

Therefore, the present invention relates to a process for the preparation of a zeolitic material having an AEI-type framework structure comprising SiO₂ and X₂O₃ in its framework structure, wherein X stands for a trivalent element, wherein said process comprises:
(1) preparing a mixture comprising one or more structure directing agents and a first zeolitic material comprising SiO₂ and X₂O₃ in its framework structure, wherein the first zeolitic material has a framework structure selected from the group consisting of FER-, TON-, MTT-, FAU-, GIS-, MOR-, BEA-, MFI-, and LTA-type framework structures, including mixtures of two or more thereof;
(2) heating the mixture obtained in (1) for obtaining a second zeolitic material having an AEI-type framework structure comprising SiO₂ and X₂O₃ in its framework structure;
(3) optionally calcining the second zeolitic material obtained in (2);
(4) optionally subjecting the zeolitic material obtained in (2) or (3) to an ion-exchange procedure, wherein preferably one or more ionic extra-framework elements contained in the zeolite framework is ion-exchanged against H⁺ and/or NH₄⁺, more preferably against NH₄⁺;
(5) calcining the zeolitic material obtained in (2), (3), or (4) at a temperature in the range of from greater than 600 to 900° C., preferably from 650 to less than 900° C., more preferably from greater than 650 to 880° C., more preferably from 700 to 870° C., more preferably from greater than 700 to 860° C., more preferably from 750 to 850° C., more preferably from 750 to less than 850° C., more preferably from 760 to 840° C., more preferably from 770 to 830° C., more preferably from 780 to 820° C., more preferably from 790 to 810° C., and more preferably from 795 to 805° C.; and
   wherein the atmosphere under which calcining of the zeolitic material in (5) is effected contains less than 10 vol.-% of H₂O, preferably 8 vol.-% or less, more preferably 5 vol.-% or less, more preferably 3 vol.-% or less, more preferably 1 vol.-% or less, more preferably 0.5 vol.-% or less, more preferably 0.1 vol.-% or less, more preferably 0.05 vol.-% or less, more preferably 0.01 vol.-% or less, more preferably 0.005 vol.-% or less, and more preferably 0.001 vol.-% or less of H₂O.

As regards the atmosphere under which calcining of the zeolitic material in (5) is effected, no particular restriction applies provided that the atmosphere under which calcining of the zeolitic material in (5) is effected contains less than 10 vol.-% of H₂O. It is preferred that the atmosphere under which calcining of the zeolitic material in (5) is effected contains less than 10 vol.-% of H₂, more preferably 8 vol.-% or less, more preferably 5 vol.-% or less, more preferably 3 vol.% or less, more preferably 1 vol.-% or less, more preferably 0.5 vol.-% or less, more preferably 0.1 vol.-% or less, more preferably 0.05 vol.-% or less, more preferably 0.01 vol.-% or less, more preferably 0.005 vol.-% or less, and more preferably 0.001 vol.-% or less of H₂.

As regards the atmosphere under which the calcining of the zeolitic material in (3) and/or (5) is effected, no particular restriction applies provided that the atmosphere under which calcining of the zeolitic material in (5) is effected contains less than 10 vol.-% of H₂O. Thus, the atmosphere under which the calcining of the zeolitic material in (3) and/or (5) is effected may comprise any combination of gaseous compounds that are suitable for calcination. It is preferred that calcining of the zeolitic material in (3) and/or (5) is effected under air as the atmosphere. More preferably, calcining of the zeolitic material in (3) and/or (5) is effected under a mixture comprising nitrogen and oxygen as the atmosphere.

As regards the conditions, e. g. the temperature, under which calcining in (3) of the second zeolitic material obtained in (2) is effected, no particular restriction applies. It is preferred that the temperature of calcination in (3) is in the range of from 400 to 850° C., more preferably from 450 to 700° C., more preferably from 550 to 650° C., and more preferably from 575 to 625° C.

As regards the conditions, e. g. the period, under which calcining in (3) and/or in (5) of the second zeolitic material obtained in (2) is effected, no particular restriction applies. It is preferred that calcining in (3) and/or (5) is conducted for a period in the range of from 0.5 to 24 h, more preferably from 1 to 16 h, more preferably from 2 to 12 h, more preferably from 2.5 to 9 h, more preferably from 3 to 7 h, more preferably from 3.5 to 6.5 h, more preferably from 4 to 6 h, and more preferably from 4.5 to 5.5 h.

Therefore, it is particularly preferred that calcining in (3) of the second zeolitic material obtained in (2) is effected under air as the atmosphere, preferably at a temperature in the range of from 400 to 850° C., more preferably from 450 to 700° C., more preferably from 550 to 650° C., and more preferably from 575 to 625° C., and preferably for a period in the range of from 0.5 to 24 h, more preferably from 1 to 16 h, more preferably from 2 to 12 h, more preferably from 2.5 to 9 h, more preferably from 3 to 7 h, more preferably from 3.5 to 6.5 h, more preferably from 4 to 6 h, and more preferably from 4.5 to 5.5 h.

Therefore, it is particularly preferred that calcining in (5) of the second zeolitic material obtained in (2), (3), or (4) is effected under an atmosphere containing less than 10 vol.-% of H₂, more preferably 8 vol.-% or less, more preferably 5 vol.-% or less, more preferably 3 vol.-% or less, more preferably 1 vol.-% or less, more preferably 0.5 vol.-% or less, more preferably 0.1 vol.-% or less, more preferably 0.05 vol.-% or less, more preferably 0.01 vol.-% or less, more preferably 0.005 vol.-% or less, and more preferably 0.001 vol.-% or less of H₂, preferably under air as the atmosphere, and preferably for a period in the range of from 0.5 to 24 h, more preferably from 1 to 16 h, more preferably from 2 to 12 h, more preferably from 2.5 to 9 h, more preferably from 3 to 7 h, more preferably from 3.5 to 6.5 h, more preferably from 4 to 6 h, and more preferably from 4.5 to 5.5 h.

As regards the conditions, e. g. the temperature, the pressure and the period, under which heating in (2) is effected, no particular restriction applies provided that the mixture is heated.

As regards the temperature at which the mixture in (2) is heated, no particular restriction applies provided that the temperature is suitable for obtaining a second zeolitic material having an AEI-type framework structure comprising SiO₂ and X₂O₃ in its framework structure. It is preferred that the mixture is heated in (2) at a temperature ranging from 90 to 250° C., more preferably from 100 to 230° C., more preferably from 110 to 210° C., more preferably from 130 to 190° C., more preferably from 140 to 180° C., more preferably from 150 to 170° C., and more preferably from 155 to 165° C.

As regards the pressure under which the heating in (2) is conducted, no particular restriction applies provided that the pressure is suitable for obtaining a second zeolitic material having an AEI-type framework structure comprising SiO₂ and X₂O₃ in its framework structure. It is preferred that the heating in (2) is conducted under autogenous pressure, more preferably under solvothermal conditions, more preferably under hydrothermal conditions. Preferably, heating in (2) is performed in a pressure tight vessel, more preferably in an autoclave.

As regards the period for which the mixture is heated in (2), no particular restriction applies provided that the pressure is suitable for obtaining a second zeolitic material having an AEI-type framework structure comprising SiO₂ and X₂O₃ in its framework structure. It is preferred that the mixture is heated for a period ranging from 0.25 to 12 d, preferably from 0.5 to 9 d, more preferably from 1 to 7 d, more preferably from 2 to 6 d, more preferably from 3 to 7 d, more preferably from 2.5 to 5.5 d, more preferably from 3 to 5 d, and more preferably from 3.5 to 4.5 d.

Therefore, it is particularly preferred that the mixture in (2) is heated at a temperature ranging from 90 to 250° C., more preferably from 100 to 230° C., more preferably from 110 to 210° C., more preferably from 130 to 190° C., more preferably from 140 to 180° C., more preferably from 150 to 170° C., and more preferably from 155 to 165° C., preferably under autogenous pressure, more preferably under solvothermal conditions, more preferably under hydrothermal conditions, and preferably for a period ranging from 0.25 to 12 d, more preferably from 0.5 to 9 d, more preferably from 1 to 7 d, more preferably from 2 to 6 d, more preferably from 3 to 7 d, more preferably from 2.5 to 5.5 d, more preferably from 3 to 5 d, and more preferably from 3.5 to 4.5 d.

As regards the atmosphere under which calcining of the zeolitic material in (3) is effected, no particular restriction applies. It is preferred that the atmosphere under which calcining of the zeolitic material in (3) is effected contains H₂ in the range of from 1 to 99 vol.-%, more preferably from 3 to 90 vol.-%, more preferably from 5 to 70 vol.-%, more preferably from 8 to 50 vol.%, more preferably from 10 to 40 vol.-%, more preferably from 13 to 30 vol.-%, more preferably from 15 to 25 vol.-%, more preferably from 17 to 23 vol.-%, and more preferably from 19 to 21 vol.-%.

In the case where the atmosphere under which calcining of the zeolitic material in (3) is effected contains H₂ in the range of from 1 to 99 vol.-%, no particular restriction applies in view of further gases that may be comprised therein. It is preferred that the hydrogen gas containing atmosphere further comprises one or more inert gases in addition to hydrogen gas, wherein more preferably the hydrogen gas containing atmosphere further comprises one or more inert gases selected from the group consisting of nitrogen, helium, neon, argon, xenon, carbon monoxide, carbon dioxide, and mixtures of two or more thereof, more preferably from the group consisting of nitrogen, argon, carbon monoxide, carbon dioxide, and mixtures of two or more thereof, wherein more preferably the hydrogen gas containing atmosphere further comprises nitrogen and/or argon, and more preferably nitrogen.

As disclosed above, no particular restriction applies in view of further gases that may be comprised in the atmosphere in the case where the atmosphere, under which calcining of the zeolitic material in (3) is effected, contains H₂ in the range of from 1 to 99 vol.-%. It is preferred that the hydrogen gas containing atmosphere contains 1 vol.-% or less of oxygen gas, more preferably 0.5 vol.-% or less, more preferably 0.1 vol.-% or less, more preferably 0.05 vol.-% or less, more preferably 0.01 vol.-% or less, more preferably 0.005 vol.-% or less, more preferably 0.001 vol.-% or less, more preferably 0.0005 vol.-% or less, and more preferably 0.0001 vol.-% or less, wherein more preferably the hydrogen gas containing atmosphere does not contain oxygen gas.

As disclosed above, the mixture prepared in (1) comprises one or more structure directing agents and a first zeolitic material comprising SiO₂ and X₂O₃ in its framework structure, wherein the first zeolitic material has a framework structure selected from the group consisting of FER-, TON-, MTT-, FAU-, GIS-, MOR-, BEA-, MFI-, and LTA-type framework structures, including mixtures of two or more thereof. As regards the molar ratio SDA:SiO₂ of the one or more structure directing agents (SDA) to SiO₂ in the framework structure of the first zeolitic material in the mixture prepared according to (1), no particular restriction applies. It is preferred that the molar ratio SDA:SiO₂ of the one or more structure directing agents (SDA) to SiO₂ in the framework structure of the first zeolitic material in the mixture prepared according to (1) ranges from 0.01 to 2, more preferably from 0.02 to 1.5, more preferably from 0.03 to 1, more preferably from 0.04 to 0.8, more preferably from 0.06 to 0.5, more preferably from 0.08 to 0.3, more preferably from 0.1 to 0.35, more preferably from 0.12 to 0.25, and more preferably from 0.15 to 0.2.

As regards the chemical and/or physical nature of the mixture prepared according to (1), no particular restriction applies such that the mixture may comprise one or more further compounds. With regard to the one or more further compounds, it is preferred that the one or more further compounds are effective as solvents. Therefore, it is preferred that the mixture prepared according to (1) further comprises one or more solvents, wherein said one or more solvents preferably comprise water, more preferably distilled water, wherein more preferably water is contained as the one or more solvents in the mixture prepared according to (1), preferably distilled water.

In the case where the mixture prepared according to (1) comprises water, no particular restriction applies as regards the molar ratio H₂O:SiO₂ of water to SiO₂ in the framework structure of the first zeolitic material in the mixture prepared according to (1). It is preferred that the molar ratio H₂O:SiO₂ of water to SiO₂ in the framework structure of the first zeolitic material in the mixture prepared according to (1) ranges from 1 to 80, more preferably from 5 to 50, more preferably from 10 to 30, and more preferably from 15 to 20.

As regards the process for the preparation of a zeolitic material having an AEI-type framework structure comprising SiO₂ and X₂O₃ in its framework structure, further process steps may be comprised therein, e. g. between (2) and (3). It is preferred that after (2) and prior to (3), the process further comprises one or more of:

(2a) isolating the zeolitic material obtained in (2), preferably by filtration, and/or

(2b) washing the zeolitic material obtained in (2) or (2a), and/or

(2c) drying the zeolitic material obtained in any of (2), (2a), or (2b).

Therefore, it is particularly preferred that the process for the preparation of a zeolitic material having an AEI-type framework structure comprising SiO₂ and X₂O₃ in its framework structure further comprises after (2) and prior to (3):

(2a) isolating the zeolitic material obtained in (2), preferably by filtration, and

(2b) washing the zeolitic material obtained in (2a), and

(2c) drying the zeolitic material obtained in (2b).

As regards the X, no particular restriction applies provided that X stands for a trivalent element. It is preferred that X is selected from the group consisting of Al, B, In, Ga, and mixtures of two or more thereof, X more preferably being Al and/or B, and more preferably being Al.

As regards the first zeolitic material comprised in the mixture prepared according to (1), no particular restriction applies provided that the first zeolitic material comprises SiO₂ and X₂O₃ in its framework structure, wherein the first zeolitic material has a framework structure selected from the group consisting of FER-, TON-, MTT-, FAU-, GIS-, MOR-, BEA-, MFI-, and LTA-type framework structures, including mixtures of two or more thereof. It is preferred that the first zeolitic material has a framework structure selected from the group consisting of FAU-, GIS-, MOR-, BEA-, MFI-, and LTA-type framework structures, including mixtures of two or more thereof, more preferably from the group consisting of FAU-, MOR-, BEA-, and MFI-type framework structures, more preferably from the group consisting of FAU-, BEA-, and MFI-type framework structures, wherein more preferably the first zeolitic material has an FAU- and/or MFI-type framework structure, wherein more preferably the first zeolitic material has an FAU-type framework structure.

As disclosed above, the first zeolitic material has a framework structure selected from the group consisting of FER-, TON-, MTT-, FAU-, GIS-, MOR-, BEA-, MFI-, and LTA-type framework structures, including mixtures of two or more thereof.

In the case where the first zeolitic material has an FAU-type framework structure, no particular restriction applies as regards the chemical and/or physical nature of the first zeolitic material. It is preferred that the first zeolitic material has an FAU-type framework structure, wherein the first zeolitic material is selected from the group consisting of ZSM-3, Faujasite, [Al—Ge—O]-FAU, CSZ1, ECR-30, Zeolite X, Zeolite Y, LZ-210, SAPO-37, ZSM-20, Na-X, US-Y, Na-Y, [Ga—Ge—O]-FAU, Li-LSX, [Ga—Al—Si—O]-FAU, and [Ga—Si—O]-FAU, including mixtures of two or more thereof, more preferably from the group consisting of ZSM-3, Faujasite, CSZ-1, ECR-30, Zeolite X, Zeolite Y, LZ-210, ZSM-20, Na-X, US-Y, Na-Y, and Li-LSX, including mixtures of two or more thereof,

more preferably from the group consisting of Faujasite, Zeolite X, Zeolite Y, Na-X, US-Y, and Na-Y, including mixtures of two or more thereof,

more preferably from the group consisting of Faujasite, Zeolite X, and Zeolite Y, including mixtures of two or more thereof,

wherein more preferably the first zeolitic material has an FAU-type framework structure and comprises zeolite X and/or zeolite Y, preferably zeolite Y,

wherein more preferably the first zeolitic material has an FAU-type framework structure and is zeolite X and/or zeolite Y, preferably zeolite Y.

In the case where the first zeolitic material has an MFI-type framework structure, no particular restriction applies as regards the chemical and/or physical nature of the first zeolitic material. It is preferred that the first zeolitic material has an MFI-type framework structure, wherein the first zeolitic material is selected from the group consisting of Silicalite, ZSM-5, [Fe—Si—O]-MFI, [Ga—SiO]-MFI, [As—Si—O]-MFI, AMS-1B, AZ-1, Bor-C, Encilite, Boralite C, FZ-1, LZ-105, Mutinaite, NU4, NU-5, TS-1, TSZ, TSZ-III, TZ-01, USC-4, USI-108, ZBH, ZKQ-1B, ZMQ-TB, MnS-1, and FeS1, including mixtures of two or more thereof,

more preferably from the group consisting of Silicalite, ZSM-5, AMS-1B, AZ-1, Encilite, FZ-1, LZ-105, Mutinaite, NU-4, NU-5, TS-1, TSZ, TSZ-III, TZ-01, USC-4, USI-108, ZBH, ZKQ-1B, and ZMQ-TB, including mixtures of two or more thereof,

wherein more preferably the first zeolitic material has an MFI-type framework structure and comprises Silicalite and/or ZSM-5, preferably ZSM-5,

wherein more preferably the first zeolitic material has an MFI-type framework structure and is zeolite Silicalite and/or ZSM-5, preferably ZSM-5.

In the case where the first zeolitic material has a BEA-type framework structure, no particular restriction applies as regards the chemical and/or physical nature of the first zeolitic material. It is preferred that the first zeolitic material has a BEA-type framework structure, wherein the first zeolitic material is selected from the group consisting of zeolite beta, Tschernichite, [B—Si—O]-*BEA, CIT-6, [Ga—Si—O]-*BEA, Beta polymorph B, SSZ-26, SSZ-33, Beta polymorph A, [Ti—Si—O]-*BEA, and pure silica beta, including mixtures of two or more thereof, more preferably from the group consisting of zeolite beta, CIT-6, Beta polymorph B, SSZ-26, SSZ-33, Beta polymorph A, and pure silica beta, including mixtures of two or more thereof, wherein more preferably the first zeolitic material having a BEA-type framework structure comprises zeolite beta, preferably zeolite beta obtained from organotemplate-free synthesis, wherein more preferably the first zeolitic material has a BEA-type framework structure and is zeolite beta, preferably zeolite beta obtained from organotemplate-free synthesis.

In the case where the first zeolitic material has a GIS-type framework structure, no particular restriction applies as regards the chemical and/or physical nature of the first zeolitic material. It is preferred that the first zeolitic material has a GIS-type framework structure, wherein the first zeolitic material is selected from the group consisting of zeolite P, TMA-gismondine, Na-P1, Amicite, Gobbinsite, High-silica Na-P, Na-P2, SAPO-43, Gismondine, MAPSO-43, MAPSO-43, Garronite, Synthetic amicite, Synthetic garronite, Synthetic gobbinsite, [Ga—Si—O]-GIS, Synthetic Ca-garronite, Low-silica Na-P (MAP), [Al—Ge—O]-GIS, including mixtures of two or more thereof, more preferably from the group consisting of zeolite P, TMA-gismondine, Na-P1, Amicite, Gobbinsite, High-silica Na-P, Na-P2, Gismondine, Garronite, Synthetic amicite, Synthetic garronite, Synthetic gobbinsite, [Ga—Si—O]-GIS, Synthetic Ca-garronite, [Al—Ge—O]-GIS, including mixtures of two or more thereof,

more preferably from the group consisting of zeolite P, TMA-gismondine, Na-P1, Amicite, Gobbinsite, High-silica Na-P, Na-P2, Gismondine, Garronite, Synthetic amicite, Synthetic garronite,

Synthetic gobbinsite, Synthetic Ca-garronite, including mixtures of two or more thereof, more preferably from the group consisting of zeolite P, Na-P1, High-silica Na-P, Na-P2, including mixtures of two or more thereof,

wherein more preferably the first zeolitic material has a GIS-type framework structure and comprises zeolite P,

wherein more preferably the first zeolitic material has a GIS-type framework structure and is zeolite P.

In the case where the first zeolitic material has an MOR-type framework structure, no particular restriction applies as regards the chemical and/or physical nature of the first zeolitic material. It is preferred that the first zeolitic material has an MOR-type framework structure, wherein the first zeolitic material is selected from the group consisting of Mordenite, [Ga—Si—O]-MOR, Maricopaite, Ca-Q, LZ-211, Na-D, RMA-1, including mixtures of two or more thereof,

wherein preferably the first zeolitic material has an MOR-type framework structure and comprises Mordenite,

wherein more preferably the first zeolitic material has an MOR-type framework structure and is Mordenite.

In the case where the first zeolitic material has an LTA-type framework structure, no particular restriction applies as regards the chemical and/or physical nature of the first zeolitic material. It is preferred that the first zeolitic material has an LTA-type framework structure, wherein the first zeolitic material is selected from the group consisting of Linde Type A (zeolite A), Alpha, [Al—Ge—O]-LTA, N-A, LZ-215, SAPO-42, ZK-4, ZK-21, Dehyd. Linde Type A (dehyd. zeolite A), ZK-22, ITQ-29, UZM-9, including mixtures of two or more thereof,

preferably from the group consisting of Linde Type A, Alpha, N-A, LZ-215, SAPO-42, ZK-4, ZK21, Dehyd. Linde Type A, ZK-22, ITQ-29, UZM-9, including mixtures of two or more thereof, more preferably from the group consisting of Linde Type A, Alpha, N-A, LZ-215, ZK-4, ZK-21, Dehyd. Linde Type A, ZK-22, ITQ-29, UZM-9, including mixtures of two or more thereof, more preferably from the group consisting of Linde Type A, Alpha, N-A, LZ-215, ZK-4, ZK-21, ZK-22, ITQ-29, UZM-9, including mixtures of two or more thereof.

As regards the chemical and/or physical nature of the second zeolitic material obtained in (2) and having an AEI-type framework structure comprising SiO₂ and X₂O₃ in its framework structure, no particular restriction applies. It is preferred that the second zeolitic material obtained in (2) having an AEI-type framework structure is selected from the group consisting of SSZ-39, SAPO-18, SIZ-8, including mixtures of two or more thereof, wherein more preferably the second zeolitic material obtained in (2) comprises SSZ-39, and wherein more preferably the second zeolitic material obtained in (2) is SSZ-39.

As regards the mixture prepared in (1) and heated in (2), no particular restriction applies provided that the mixture comprises one or more structure directing agents and a first zeolitic material comprising SiO₂ and X₂O₃ in its framework structure, wherein the first zeolitic material has a framework structure selected from the group consisting of FER-, TON-, MTT-, FAU-, GIS-, MOR-, BEA-, MFI-, and LTA-type framework structures, including mixtures of two or more thereof and further provided that a second zeolitic material having an AEI-type framework structure comprising SiO₂ and X₂O₃ in its framework structure can be obtained upon heating the mixture obtained in (1). Therefore, the mixture prepared in (1) and heated in (2) may contain further compounds, e.g. at least one source for OH or OH as such. It is preferred the mixture prepared in (1) and heated in (2) further comprises at least one source for OH, wherein said at least one source for OH preferably comprises a metal hydroxide, more preferably a hydroxide of an alkali metal M, more preferably sodium and/or potassium hydroxide, and more preferably sodium hydroxide, wherein more preferably the at least one source for OH is sodium hydroxide.

In the case where the mixture prepared in (1) and heated in (2), as disclosed above, comprises at least one source for OH⁻, no particular restriction applies in view of the OH⁻:SiO₂ molar ratio of OH⁻ to SiO₂ in the framework structure of the first zeolitic material in the mixture prepared according to (1) provided that a second zeolitic material having an AEI-type framework structure comprising SiO₂ and X₂O₃ in its framework structure can be obtained upon heating the mixture obtained in (1). It is preferred that the OH⁻:SiO₂ molar ratio of OH to SiO₂ in the framework structure of the first zeolitic material in the mixture prepared according to (1) is in the range of from 0.01 to 1, more preferably from 0.03 to 0.7, more preferably from 0.05 to 0.5, more preferably from 0.1 to 0.45, more preferably from 0.15 to 0.4, more preferably from 0.2 to 0.35, and more preferably from 0.25 to 0.3.

As disclosed above the process of the present invention comprises one or more structure directing agents in the mixture in (1). As regards the physical and/or chemical nature of the one or more structure directing agents in the mixture in (1) no particular restriction applies provided that a second zeolitic material having an AEI-type framework structure comprising SiO₂ and X₂O₃ in its framework structure can be obtained upon heating the mixture obtained in (1). According to a first alternative, it is preferred that the one or more structure directing agents comprises one or more tetraalkylammonium cation R¹R²R³R⁴N⁺-containing compounds, wherein R¹, R², R³ and R⁴ independently from one another stand for alkyl, and wherein R³ and R⁴ form a common alkyl chain.

In the case where the one or more structure directing agents comprises one or more tetraalkylammonium cation R¹R²R³R⁴N⁺-containing compounds as disclosed above, no particular restriction applies in view of R¹ and R² provided that R¹ and R² independently from one another stand for alkyl. It is preferred that R¹ and R² independently from one another stand for optionally substituted and/or optionally branched (C₁-C₆)alkyl, more preferably (C₁-C₅)alkyl, more preferably (C₁-C₄)alkyl, more preferably (C₁-C₃)alkyl, and more preferably for optionally substituted methyl or ethyl, wherein more preferably R¹ and R² independently from one another stand for optionally substituted methyl or ethyl, preferably unsubstituted methyl or ethyl, wherein more preferably R¹ and R² independently from one another stand for optionally substituted methyl, preferably unsubstituted methyl.

In the case where the one or more structure directing agents comprises one or more tetraalkylammonium cation R¹R²R³R⁴N⁺-containing compounds as disclosed above, no particular restriction applies in view of R³ and R⁴ provided that R³ and R⁴ independently from one another stand for alkyl, and wherein R³ and R⁴ form a common alkyl chain. It is preferred that R³ and R⁴ form a common derivatized or underivatized, preferably underivatized alkyl chain, more preferably a common (C₄-C₈)alkyl chain, more preferably a common (C₄-C₇)alkyl chain, more preferably a common (C₄-C₆)alkyl chain, wherein more preferably said common alkyl chain is a derivatized or underivatized, preferably underivatized C4 or C5 alkyl chain, and more preferably a derivatized or underivatized, preferably underivatized C5 alkyl chain.

Therefore, it is particularly preferred that in the case where the one or more structure directing agents comprises one or more tetraalkylammonium cation R¹R²R³R⁴N⁺-containing compounds, preferably as disclosed above, the one or more structure directing agents comprises one or more tetraalkylammonium cation R¹R²R³R⁴N⁺-containing compounds, wherein R¹, R², R³ and R⁴ independently from one another stand for alkyl, and wherein R³ and R⁴ form a common alkyl chain, that R¹ and R² independently from one another stand for optionally substituted and/or optionally branched (C₁-C₆)alkyl, more preferably (C₁-C₅)alkyl, more preferably (C₁-C₄)alkyl, more preferably (C₁-C₃)alkyl, and more preferably for optionally substituted methyl or ethyl, wherein more preferably R¹ and R² independently from one another stand for optionally substituted methyl or ethyl, preferably unsubstituted methyl or ethyl, wherein more preferably R¹ and R² independently from one another stand for optionally substituted methyl, preferably unsubstituted methyl, and that R³ and R⁴ form a common derivatized or underivatized, preferably underivatized alkyl chain, more preferably a common (C₄-C₈)alkyl chain, more preferably a common (C₄-C₇)alkyl chain, more preferably a common (C₄-C₆)alkyl chain, wherein more preferably said common alkyl chain is a derivatized or underivatized, preferably underivatized C4 or C5 alkyl chain, and more preferably a derivatized or underivatized, preferably underivatized C5 alkyl chain.

In the case where the one or more structure directing agents comprises one or more tetraalkylammonium cation R¹R²R³R⁴N⁺-containing compounds as disclosed above, no particular restriction applies in view of the physical and/or chemical nature of the ammonium compounds comprised therein. It is preferred that the one or more tetraalkylammonium cation R¹R²R³R⁴N⁺-containing compounds comprise one or more ammonium compounds selected from the group consisting of derivatized or underivatized, preferably underivatized N,N-di(C₁-C₄)alkyl-3,5-di(C₁-C₄)alkylpyrrolidinium compounds, N,N-di(C₁-C₄)alkyl-3,5-di(C₁-C₄)alkylpiperidinium compounds, N,N-di(C₁-C₄)alkyl-3,5-di(C₁-C₄)alkylhexahydroazepinium compounds, N,N-di(C₁-C₄)alkyl-2,6-di(C₁-C₄)alkylpyrrolidinium compounds, N,N-di(C₁-C₄)alkyl-2,6-di(C₁-C₄)alkylpiperidinium compounds, N,N-di(C₁-C₄)alkyl-2,6-di(C₁-C₄)alkylhexahydroazepinium compounds, and mixtures of two or more thereof,

more preferably from the group consisting of N,N-di(C₁-C₄)alkyl-3,5-di(C₁-C₄)alkylpyrrolidinium compounds, N,N-di(C₁-C₄)alkyl-3,5-di(C₁-C₄)alkylpiperidinium compounds, N,N-di(C₁-C₄)alkyl-3,5-di(C₁-C₄)alkylhexahydroazepinium compounds, N,N-di(C₁-C₄)alkyl-2,6-di(C₁-C₄)alkylpyrrolidinium compounds, N,N-di(C₁-C₄)alkyl-2,6-di(C₁-C₄)alkylpiperidinium compounds, N,N-di(C₁-C₄)alkyl-2,6-di(C₁-C₄)alkylhexahydroazepinium compounds, and mixtures of two or more thereof,

more preferably from the group consisting of N,N-di(C₁-C₃)alkyl-3,5-di(C₁-C₃)alkylpyrrolidinium compounds, N,N-di(C₁-C₃)alkyl-3,5-di(C₁-C₃)alkylpiperidinium compounds, N,N-di(C₁-C₃)alkyl-3,5-di(C₁-C₃)alkylhexahydroazepinium compounds, N,N-di(C₁-C₃)alkyl-2,6-di(C₁-C₃)alkylpyrrolidinium compounds, N,N-di(C₁-C₃)alkyl-2,6-di(C₁-C₃)alkylpiperidinium compounds, N,N-di(C₁-C₃)alkyl-2,6-di(C₁-C₃)alkylhexahydroazepinium compounds, and mixtures of two or more thereof,

more preferably from the group consisting of N,N-di(C₁-C₂)alkyl-3,5-di(C₁-C₂)alkylpyrrolidinium compounds, N,N-di(C₁-C₂)alkyl-3,5-di(C₁-C₂)alkylpiperidinium compounds, N,N-di(C₁-C₂)alkyl-3,5-di(C₁-C₂)alkylhexahydroazepinium compounds, N,N-di(C₁-C₂)alkyl-2,6-di(C₁-C₂)alkylpyrrolidinium compounds, N,N-di(C₁-C₂)alkyl-2,6-di(C₁-C₂)alkylpiperidinium compounds, N,N-di(C₁-C₂)alkyl-2,6-di(C₁-C₂)alkylhexahydroazepinium compounds, and mixtures of two or more thereof,

more preferably from the group consisting of N,N-di(C₁-C₂)alkyl-3,5-di(C₁-C₂)alkylpiperidinium compounds, N,N-di(C₁-C₂)alkyl-2,6-di(C₁-C₂)alkylpiperidinium compounds, and mixtures of two or more thereof, wherein more preferably the one or more tetraalkylammonium cation R¹R²R³R⁴N⁺-containing compounds comprise one or more N,N-dimethyl-3,5-dimethylpiperidinium and/or N,N-dimethyl-2,6-dimethylpiperidinium compounds, preferably one or more N,N-dimethyl-3,5-dimethylpiperidinium compounds.

In the case where the one or more structure directing agents comprises one or more tetraalkylammonium cation R¹R²R³R⁴N⁺-containing compounds as disclosed above, no particular restriction applies in view of the physical and/or chemical nature of the ammonium compounds comprised therein. It is preferred that the one or more tetraalkylammonium cation R¹R²R³R⁴N⁺-containing compounds are salts. More preferably, the one or more tetraalkylammonium cation R¹R²R³R⁴N⁺-containing compounds are one or more salts selected from the group consisting of halides, sulfate, nitrate, phosphate, acetate, and mixtures of two or more thereof, more preferably from the group consisting of bromide, chloride, hydroxide, sulfate, and mixtures of two or more thereof, wherein more preferably the one or more tetraalkylammonium cation R¹R²R³R⁴N⁺-containing compounds are tetraalkylammonium hydroxides and/or bromides, and more preferably tetraalkylammonium hydroxides.

In the case where the one or more structure directing agents comprises one or more tetraalkylammonium cation R¹R²R³R⁴N⁺-containing compounds as disclosed above, no particular restriction applies in view of further compounds that may be comprised in the mixture prepared according to (1). It is preferred that the mixture prepared according to (1) further comprises distilled water, wherein the molar ratio H₂O:SiO₂ of water to SiO₂ in the framework structure of the first zeolitic material in the mixture prepared according to (1) ranges from 1 to 80, more preferably from 5 to 60, more preferably from 10 to 50, more preferably from 15 to 45, more preferably from 20 to 40, more preferably from 25 to 35, and more preferably from 28 to 32.

Further, in the case where the one or more structure directing agents comprises one or more tetraalkylammonium cation R¹R²R³R⁴N⁺-containing compounds as disclosed above, no particular restriction applies in view of the molar ratio R¹R²R³R⁴N⁺: SiO₂ of the one or more tetraalkylammonium cations to SiO₂ in the framework structure of the first zeolitic material in the mixture prepared according to (1). It is preferred that the molar ratio R¹R²R³R⁴N⁺: SiO₂ of the one or more tetraalkylammonium cations to SiO₂ in the framework structure of the first zeolitic material in the mixture prepared according to (1) ranges from 0.01 to 1.5, more preferably from 0.05 to 1, more preferably from 0.1 to 0.8, more preferably from 0.3 to 0.5, more preferably from 0.5 to 0.3, more preferably from 0.8 to 0.25, more preferably from 0.1 to 0.2, more preferably from 0.12 to 0.18, and more preferably from 0.14 to 0.16.

Further, in the case where the one or more structure directing agents comprises one or more tetraalkylammonium cation R¹R²R³R⁴N⁺-containing compounds as disclosed above, no particular restriction applies in view of the SiO₂:X₂O₃ molar ratio of the framework structure of the first zeolitic material. It is preferred that the framework structure of the first zeolitic material displays an SiO₂:X₂O₃ molar ratio ranging from 1 to 50, more preferably from 2 to 25, more preferably from 3.5 to 15, more preferably from 3 to 10, more preferably from 4.5 to 8, and more preferably from 5 to 6.

Further, in the case where the one or more structure directing agents comprises one or more tetraalkylammonium cation R¹R²R³R⁴N⁺-containing compounds as disclosed above, no particular restriction applies in view of further compounds that may be comprised in the mixture prepared in (1) and heated in (2). It is preferred that the mixture prepared in (1) and heated in (2) further comprises at least one source for OH⁻, wherein the OH⁻:SiO₂ molar ratio of OH⁻ to SiO₂ in the framework structure of the first zeolitic material in the mixture prepared according to (1) is in the range of from 0.1 to 1, more preferably from 0.3 to 0.7, more preferably from 0.4 to 0.5, and more preferably from 0.43 to 0.48.

As disclosed above the process of the present invention comprises one or more structure directing agents in the mixture in (1). As regards the physical and/or chemical nature of the one or more structure directing agents in the mixture in (1) no particular restriction applies provided that a second zeolitic material having an AEI-type framework structure comprising SiO₂ and X₂O₃ in its framework structure can be obtained upon heating the mixture obtained in (1). According to a second alternative, it is preferred that the one or more structure directing agents comprises one or more structure directing agents comprises one or more quaternary phosphonium cation R¹R²R³R⁴P⁺-containing compounds, wherein R¹, R², R³, and R⁴ independently from one another stand for optionally substituted and/or optionally branched (C₁-C₆)alkyl, more preferably (C₁-C₅)alkyl, more preferably (C₁-C₄)alkyl, more preferably (C₂-C₃)alkyl, and more preferably for optionally substituted methyl or ethyl, wherein more preferably R¹, R², R³, and R⁴ stand for optionally substituted ethyl, preferably unsubstituted ethyl.

In the case where the one or more structure directing agents comprises one or more quaternary phosphonium cation R¹R²R³R⁴P⁺-containing compounds as disclosed above, no particular restriction applies in view of the physical and/or chemical nature of the ammonium compounds comprised therein. It is preferred that the one or more quaternary phosphonium cation R¹R²R³R⁴P⁺-containing compounds are salts, more preferably one or more salts selected from the group consisting of halides, preferably chloride and/or bromide, more preferably chloride, hydroxide, sulfate, nitrate, phosphate, acetate, and mixtures of two or more thereof, more preferably from the group consisting of chloride, hydroxide, sulfate, and mixtures of two or more thereof, wherein more preferably the one or more quaternary phosphonium cation containing compounds are hydroxides and/or chlorides, and more preferably hydroxides.

In the case where the one or more structure directing agents comprises one or more quaternary phosphonium cation R¹R²R³R⁴P⁺-containing compounds as disclosed above, no particular restriction applies in view of further compounds, e. g. water or distilled water, that may be comprised in the mixture prepared according to (1). It is preferred that the mixture prepared according to (1) further comprises distilled water, wherein the molar ratio H₂O:SiO₂ of water to SiO₂ in the framework structure of the first zeolitic material in the mixture prepared according to (1) ranges from 1 to 80, more preferably from 1.5 to 50, more preferably from 2 to 30, more preferably from 2.5 to 15, more preferably from 3 to 10, more preferably from 3.5 to 8, more preferably from 4 to 6, and more preferably from 4.5 to 5.5.

In the case where the one or more structure directing agents comprises one or more quaternary phosphonium cation R¹R²R³R⁴P⁺-containing compounds as disclosed above, no particular restriction applies in view of the molar ratio R¹R²R³R⁴P⁺: SiO₂ of the one or more quaternary phosphonium cations to SiO₂ in the framework structure of the first zeolitic material in the mixture prepared according to (1). It is preferred that the molar ratio R¹R²R³R⁴P⁺: SiO₂ of the one or more quaternary phosphonium cations to SiO₂ in the framework structure of the first zeolitic material in the mixture prepared according to (1) ranges from 0.01 to 2, more preferably from 0.05 to 1.5, more preferably from 0.1 to 1, more preferably from 0.3 to 0.8, more preferably from 0.5 to 0.5, more preferably from 0.8 to 0.4, more preferably from 0.1 to 0.35, more preferably from 0.12 to 0.3, more preferably from 0.15 to 0.25, more preferably from 0.17 to 0.23, and more preferably from 0.19 to 0.21.

Further, in the case where the one or more structure directing agents comprises one or more quaternary phosphonium cation R¹R²R³R⁴P⁺-containing compounds as disclosed above, no particular restriction applies in view of the SiO₂:X₂O₃ molar ratio of the framework structure of the first zeolitic material. It is preferred that the framework structure of the first zeolitic material displays an SiO₂:X₂O₃ molar ratio ranges from 1 to 150, more preferably from 5 to 100, more preferably from 10 to 70, more preferably from 15 to 50, more preferably from 20 to 40, more preferably from 25 to 35, and more preferably from 28 to 32.

Further, in the case where the one or more structure directing agents comprises one or more quaternary phosphonium cation R¹R²R³R⁴P⁺-containing compounds as disclosed above, no particular restriction applies in view of further compounds, e. g. at least one source for OH⁻ or OH⁻ as such, that may be comprised in the mixture prepared in (1) and heated in (2). It is preferred that the mixture prepared in (1) and heated in (2) further comprises at least one source for OH⁻. Preferably, the mixture prepared in (1) and heated in (2) further comprises at least one source for OH⁻ and the OH⁻:SiO₂ molar ratio of OH⁻ to SiO₂ in the framework structure of the first zeolitic material in the mixture prepared according to (1) is in the range of from 0.01 to 0.3, more preferably from 0.03 to 0.2, more preferably from 0.05 to 0.15, and more preferably from 0.08 to 0.12.

Further, the present invention relates to a zeolitic material having an AEI-type framework structure obtainable and/or obtained according to the process as disclosed herein.

Further, the present invention relates to a zeolitic material having an AEI-type framework structure comprising SiO₂ and X₂O₃ in its framework structure, preferably obtainable and/or obtained according to the process as disclosed herein, wherein X stands for a trivalent element, and wherein the deconvoluted ammonia temperature programmed desorption spectrum of the zeolitic material displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g. According to the present invention the ammonia temperature programmed desorption is preferably performed and the results evaluated as described in the experimental section.

As regards the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies provided that it displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g. It is preferred that peak I is in the range of from 208 to 260° C., more preferably from 210 to 240° C., more preferably from 212 to 235° C., more preferably from 213 to 230° C., more preferably from 214 to 225° C., more preferably from 215 to 220° C., and more preferably from 216 to 218° C., wherein more preferably peak I is at 217° C.

Further, as regards the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies provided that it displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g. It is preferred that the integration of peak I affords an amount of acid sites in the range of from 0.09 to 0.3 mmol/g, more preferably from 0.11 to 0.25 mmol/g, more preferably from 0.12 to 0.2 mmol/g, more preferably from 0.125 to 0.17 mmol/g, more preferably from 0.13 to 0.15 mmol/g.

Therefore, it is particularly preferred that the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g, preferably peak I is in the range of from 208 to 260° C., more preferably from 210 to 240° C., more preferably from 212 to 235° C., more preferably from 213 to 230° C., more preferably from 214 to 225° C., more preferably from 215 to 220° C., and more preferably from 216 to 218° C., wherein more preferably peak I is at 217° C., wherein preferably the integration of peak I affords an amount of acid sites in the range of from 0.09 to 0.3 mmol/g, more preferably from 0.11 to 0.25 mmol/g, more preferably from 0.12 to 0.2 mmol/g, more preferably from 0.125 to 0.17 mmol/g, more preferably from 0.13 to 0.15 mmol/g.

As regards the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies provided that it displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g. It is preferred that peak II is in the range of from 310 to 430° C., more preferably from 315 to 400° C., more preferably from 320 to 380° C., more preferably from 325 to 360° C., more preferably from 330 to 350° C., more preferably from 333 to 345° C., and more preferably from 335 to 340° C.

Further, as regards the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies provided that it displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g. It is preferred that the integration of peak II affords an amount of acid sites in the range of from 0.28 to 0.37 mmol/g, preferably from 0.3 to 0.35 mmol/g, more preferably from 0.31 to 0.34 mmol/g, and more preferably from 0.32 to 0.33 mmol/g.

Therefore, it is particularly preferred that the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g, preferably peak II is in the range of from 310 to 430° C., more preferably from 315 to 400° C., more preferably from 320 to 380° C., more preferably from 325 to 360° C., more preferably from 330 to 350° C., more preferably from 333 to 345° C., and more preferably from 335 to 340° C., wherein preferably the integration of peak I affords an amount of acid sites in the range of from 0.07 to 0.35 mmol/g, and the integration of peak II affords an amount of acid sites in the range of from 0.25 to 0.4 mmol/g. It is preferred that the integration of peak II affords an amount of acid sites in the range of from 0.28 to 0.37 mmol/g, preferably from 0.3 to 0.35 mmol/g, more preferably from 0.31 to 0.34 mmol/g, and more preferably from 0.32 to 0.33 mmol/g.

As regards the ratio of the amount of acid sites from the integration of peak I to the amount of acid sites from the integration of peak II of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies. It is preferred that the ratio of the amount of acid sites from the integration of peak I to the amount of acid sites from the integration of peak II is in the range of from 0.35 to 0.7, more preferably from 0.38 to 0.6, more preferably from 0.4 to 0.5, more preferably from 0.41 to 0.47, more preferably from 0.42 to 0.45, and more preferably from 0.43 to 0.44.

Further, as regards the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies in view of one or more further peaks, e. g. a third peak, comprised therein. It is preferred that the deconvoluted ammonia temperature programmed desorption spectrum of the zeolitic material further displays a third peak (peak III) in the range of from 160 to 177° C., wherein the integration of peak III affords an amount of acid sites in the range of from 0.05 to 0.35 mmol/g.

Further, as regards the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, no particular restriction applies in view of one or more further peaks, e. g. a third peak, comprised therein. It is preferred that the deconvoluted ammonia temperature programmed desorption spectrum of the zeolitic material further displays a third peak (peak III) in the range of from 160 to 177° C., preferably from 163 to 174° C., more preferably from 165 to 172° C., more preferably from 166 to 171° C., more preferably from 167 to 170° C., and more preferably from 168 to 169° C.

In the case where the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, displays a third peak (peak III), no particular restriction applies in view of the integration of peak III. It is preferred that the integration of peak III affords an amount of acid sites in the range of from 0.07 to 0.3 mmol/g, more preferably from 0.09 to 0.25 mmol/g, more preferably from 0.1 to 0.2 mmol/g, more preferably from 0.11 to 0.17 mmol/g, more preferably from 0.11 to 0.15 mmol/g, more preferably from 0.12 to 0.14 mmol/g, and more preferably from 0.12 to 0.13 mmol/g.

Therefore, it is particularly preferred that the deconvoluted ammonia temperature programmed desorption spectrum of the zeolitic material having an AEI-type framework structure comprising SiO₂ and X₂O₃ in its framework structure, wherein X stands for a trivalent element, preferably obtainable and/or obtained according to the process as disclosed herein, displays a third peak (peak III), preferably in the range of from 160 to 177° C., more preferably from 163 to 174° C., more preferably from 165 to 172° C., more preferably from 166 to 171° C., more preferably from 167 to 170° C., and more preferably from 168 to 169° C., wherein the integration of peak III affords an amount of acid sites in the range of from 0.05 to 0.35 mmol/g, more preferably of from 0.07 to 0.3 mmol/g, more preferably from 0.09 to 0.25 mmol/g, more preferably from 0.1 to 0.2 mmol/g, more preferably from 0.11 to 0.17 mmol/g, more preferably from 0.11 to 0.15 mmol/g, more preferably from 0.12 to 0.14 mmol/g, and more preferably from 0.12 to 0.13 mmol/g.

As regards the inventive zeolitic material, it is further preferred that the the CO-FTIR spectrum thereof displays a first peak in the range of from 3290 to 3315 cm⁻¹ and a second peak in the range of from 3420 to 3470 cm⁻¹, wherein the maximum absorbance of the second peak is equal to or greater than the maximum absorbance of the first peak. Concerning the first peak in the CO-FTIR spectrum of the inventive zeolitic material, it is further preferred that it is in the range of from 3290 to 3315 cm⁻¹, and more preferably from 3295 to 3310 cm⁻¹, more preferably from 3300 to 3306 cm⁻¹, more preferably from 3301 to 3305 cm⁻¹, and more preferably from 3302 to 3304 cm⁻¹. With respect to the second peak in the CO-FTIR spectrum of the inventive zeolitic material, it is further preferred that it is in the range of from, and more preferably from 3425 to 3465 cm⁻¹, more preferably from 3430 to 3460 cm⁻¹, more preferably from 3435 to 3456 cm⁻¹, more preferably from 3437 to 3453 cm⁻¹, and more preferably from 3439 to 3451 cm⁻¹. Finally, with regard to the maximum absorbance of the second peak being equal to or greater than the maximum absorbance of the first peak, it is further preferred that the maximum absorbance of the second peak is greater than the maximum absorbance of the first peak.

With regard to the measurement of the CO-FTIR spectrum, no particular restrictions apply as to how it is determined, wherein it is preferred according to the present invention that the CO-FTIR spectrum is determined according to the procedure described in the experimental section of the present application.

Further, as regards the zeolitic material having an AEI-type framework structure comprising SiO₂ and X₂O₃ in its framework structure, wherein X stands for a trivalent element, preferably obtainable and/or obtained according to the process as disclosed herein, no particular restriction applies in view of the SiO₂:X₂O₃ molar ratio of SiO₂ to X₂O₃ respectively in the framework structure of the zeolitic material. It is preferred that the SiO₂:X₂O₃ molar ratio of SiO₂ to X₂O₃ respectively in the framework structure of the zeolitic material is in the range of from 2 to 150, more preferably of from 4 to 100, more preferably of from 8 to 50, more preferably of from 12 to 35, more preferably of from 16 to 30, more preferably of from 18 to 26, and more preferably of from 20 to 24.

Further, as regards the zeolitic material having an AEI-type framework structure comprising SiO₂ and X₂O₃ in its framework structure, preferably obtainable and/or obtained according to the process as disclosed herein, no particular restriction applies in view of X comprised therein provided that X stands for a trivalent element. It is preferred that X is selected from the group consisting of Al, B, In, Ga, and mixtures of two or more thereof, X preferably being Al and/or B, and more preferably being Al.

Further, as regards the zeolitic material having an AEI-type framework structure comprising SiO₂ and X₂O₃ in its framework structure, wherein X stands for a trivalent element, preferably obtainable and/or obtained according to the process as disclosed herein, no particular restriction applies in view of the chemical and/or physical properties, e. g. the BET surface area, of the zeolitic material. It is preferred that the BET surface area of the zeolitic material is in the range of from 400 to 800 m²/g, more preferably of from 450 to 750 m²/g, more preferably of from 500 to 700 m²/g, more preferably of from 550 to 680 m²/g, more preferably of from 600 to 670 m²/g, and more preferably of from 630 to 660 m²/g, wherein the BET surface area of the zeolitic material is preferably determined according to ISO 9277:2010. Alternatively, it is preferred that the BET surface area is determined according to the procedure described in the experimental section.

As disclosed above, no particular restriction applies in view of the chemical and/or physical properties of the zeolitic material having an AEI-type framework structure comprising SiO₂ and X₂O₃ in its framework structure, wherein X stands for a trivalent element, preferably obtainable and/or obtained according to the process as disclosed herein. It is preferred that the micropore volume of the zeolitic material is in the range of from 0.1 to 0.3 cm³/g, more preferably of from 0.13 to 0.26 cm³/g, more preferably of from 0.15 to 0.24 cm³/g, more preferably of from 0.17 to 0.22 cm³/g, and more preferably of from 0.19 to 0.21 cm³/g, wherein the micropore volume of the zeolitic material is preferably determined according to DIN 66135-3:2001-06. Alternatively, it is preferred that the micropore volume is determined according to the procedure described in the experimental section.

As disclosed above, no particular restriction applies in view of the chemical and/or physical properties of the zeolitic material having an AEI-type framework structure comprising SiO₂ and X₂O₃ in its framework structure, wherein X stands for a trivalent element, preferably obtainable and/or obtained according to the process as disclosed herein. It is preferred that the total pore volume of the zeolitic material is in the range of from 0.35 to 0.55 cm³/g, preferably of from 0.38 to 0.48 cm³/g, more preferably of from 0.4 to 0.45 cm³/g, and more preferably of from 0.41 to 0.42 cm³/g, wherein the total pore volume of the zeolitic material is preferably determined according to ISO 9277:2010. Alternatively, it is preferred that the total micropore volume is determined according to the procedure described in the experimental section.

As regards the zeolitic material itself having an AEI-type framework structure comprising SiO₂ and X₂O₃ in its framework structure, wherein X stands for a trivalent element, preferably obtainable and/or obtained according to the process as disclosed herein, no particular restriction applies. It is preferred that the zeolitic material having an AEI-type framework structure comprising SiO₂ and X₂O₃ in its framework structure, wherein X stands for a trivalent element, preferably obtainable and/or obtained according to the process as disclosed herein, is selected from the group consisting of SSZ-39, SAPO-18, and SIZ-8, including mixtures of two or more thereof, wherein more preferably the zeolitic material comprises SSZ-39, and wherein more preferably the zeolitic material is SSZ-39.

Further, the present invention relates to a process for the conversion of oxygenates to olefins, wherein the process comprises
(I) providing a gas stream comprising one or more oxygenates;
(II) contacting the gas stream with a catalyst comprising a zeolitic material as disclosed herein.

As regards the one or more oxygenates contained in the gas stream provided in step (I) of the process, no particular restriction applies. It is preferred that the gas stream provided in step (I) contains one or more oxygenates selected from the group consisting of aliphatic alcohols, ethers, carbonyl compounds, and mixtures of two or more thereof, more preferably from the group consisting of C₁-C₆-alcohols, di-C₁-C₃-alkyl ethers, C₁-C₆-aldehydes, C₂-C₆-ketones, and mixtures of two or more thereof, more preferably from the group consisting of C₁-C₄-alcohols, di-C₁-C₂-alkyl ethers, C₁-C₄-aldehydes, C₂-C₄-ketones, and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone, and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone, and mixtures of two or more thereof, more preferably from the group consisting of methanol, ethanol, dimethyl ether, diethyl ether, ethyl methyl ether, and mixtures of two or more thereof, wherein more preferably the gas stream provided in step (I) comprises methanol and/or dimethyl ether, preferably methanol.

As regards the amount of the one or more oxygenates contained in the gas stream provided in step (I), no particular restriction applies. It is preferred that the gas stream provided in step (I) contains the one or more oxygenates in an amount in the range of from 30 to 100 vol.-% of based on the total volume of the gas stream, more preferably from 30 to 99.9 vol.-%, more preferably from 30 to 99 vol.-%, more preferably from 30 to 95 vol.-%, more preferably from 30 to 90 vol.-%, more preferably from 30 to 80 vol.-%, more preferably from 30 to 70 vol.-%, more preferably from 30 to 60 vol.-%, more preferably from 30 to 50 vol.-%, and more preferably from 30 to 45 vol.-%.

As regards the gas stream provided in step (I), no particular restriction applies such that further compounds, e. g. water, may be comprised therein. According to a first alternative, It is preferred that the gas stream provided in step (I) contains 60 vol.-% or less of H₂O based on the total volume of the gas stream, wherein preferably the gas stream provided in step (I) contains H₂O in the range of from 5 to 60 vol.-%, more preferably from 10 to 55 vol.-%, more preferably from 20 to 50 vol.-%, and more preferably from 30 to 45 vol.-%.

As disclosed above, no particular restriction applies to the gas stream provided in (1) such that further compounds, e. g. water, may be comprised therein. According to a second alternative, it is preferred that the gas stream provided in step (I) contains 5 vol.-% or less of H₂O based on the total volume of the gas stream, preferably 3 vol.-% or less, more preferably 1 vol.-% or less, more preferably 0.5 vol.-% or less, more preferably 0.1 vol.-% or less, more preferably 0.05 vol.% or less, more preferably 0.01 vol.-% or less, more preferably 0.005 vol.-% or less, and more preferably 0.001 vol.-% or less.

As regards the conditions, e. g. the temperature and/or the period, under which contacting of the gas stream with the catalyst in step (II) is performed, no particular restriction applies. It is preferred that contacting of the gas stream with the catalyst in step (II) is performed at a temperature in the range of from 200 to 700° C., more preferably from 250 to 650° C., more preferably from 300 to 600° C., more preferably from 350 to 560° C., more preferably from 400 to 540° C., more preferably from 430 to 520° C., and more preferably from 450 to 500° C.

As disclosed above, no particular restriction applies in view of the conditions, e. g. the temperature and/or the period, under which contacting of the gas stream with the catalyst in step (II) is performed. It is preferred that contacting of the gas stream with the catalyst in step (II) is performed at a pressure in the range of from 0.1 to 10 bar, preferably from 0.3 to 7 bar, more preferably from 0.5 to 5 bar, more preferably from 0.7 to 3 bar, more preferably from 0.8 to 2.5 bar, more preferably from 0.9 to 2.2 bar, and more preferably from 1 to 2 bar. According to the present invention, the pressure as defined in the present application designates the absolute pressure such that a pressure of 1 bar upon contacting of the gas stream with the catalyst corresponds to the normal pressure of 1.03 kPa.

Therefore, it is particularly preferred that contacting of the gas stream with the catalyst in step (II) is performed at a temperature in the range of from 200 to 700° C., more preferably from 250 to 650° C., more preferably from 300 to 600° C., more preferably from 350 to 560° C., more preferably from 400 to 540° C., more preferably from 430 to 520° C., and more preferably from 450 to 500° C., and at a pressure in the range of from 0.1 to 10 bar, preferably from 0.3 to 7 bar, more preferably from 0.5 to 5 bar, more preferably from 0.7 to 3 bar, more preferably from 0.8 to 2.5 bar, more preferably from 0.9 to 2.2 bar, and more preferably from 1 to 2 bar.

As regards the mode in which the process for the conversion of oxygenates to olefins as disclosed herein is performed, no particular restriction applies. It is preferred that the process is performed as a batch process or in a continuous mode, wherein more preferably the process is performed at least in part in a continuous mode, wherein more preferably the process is performed in a continuous mode.

In the case where the process is performed in a continuous mode, no particular restriction applies in view of the weight hourly space velocity (WHSV) of the gas stream in step (II). It is preferred that the weight hourly space velocity (WHSV) of the gas stream in step (II) is in the range of from 0.5 to 50 h⁻¹, preferably from 1 to 30 h⁻¹, more preferably from 2 to 20 h⁻¹, more preferably from 3 to 15 h⁻¹, more preferably from 4 to 10 h⁻¹, and more preferably from 5 to 7 h⁻¹.

Further, the present invention relates to a use of a zeolitic material as disclosed herein as a molecular sieve, catalyst, catalyst support, and/or as an adsorbent, preferably as a catalyst and/or as a catalyst support for the selective catalytic reduction (SCR) of nitrogen oxides NO_{R}; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, more preferably as a catalyst and/or as a catalyst support in the conversion of alcohols to olefins, and more preferably as a catalyst for the conversion of alcohols to olefins, preferably of methanol to olefins.

The present invention is further illustrated by the following embodiments and combinations of embodiments as indicated by the respective dependencies and back-references. In particular, it is noted that in each instance where a combination of embodiments is mentioned as a range, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Thus, the present invention includes the following embodiments, wherein these include the specific combinations of embodiments as indicated by the respective interdependencies defined therein:
1. A process for the preparation of a zeolitic material having an AEI-type framework structure comprising SiO₂ and X₂O₃ in its framework structure, wherein X stands for a trivalent element, wherein said process comprises:
   (1) preparing a mixture comprising one or more structure directing agents and a first zeolitic material comprising SiO₂ and X₂O₃ in its framework structure, wherein the first zeolitic material has a framework structure selected from the group consisting of FER-, TON-, MTT-, FAU-, GIS-, MOR-, BEA-, MFI-, and LTA-type framework structures, including mixtures of two or more thereof;
   (2) heating the mixture obtained in (1) for obtaining a second zeolitic material having an AEI-type framework structure comprising SiO₂ and X₂O₃ in its framework structure;
   (3) optionally calcining the second zeolitic material obtained in (2);
   (4) optionally subjecting the zeolitic material obtained in (2) or (3) to an ion-exchange procedure, wherein preferably one or more ionic extra-framework elements contained in the zeolite framework is ion-exchanged against H⁺ and/or NH₄⁺, more preferably against NH₄⁺;
   (5) calcining the zeolitic material obtained in (2), (3), or (4) at a temperature in the range of from greater than 600 to 900° C., preferably from 650 to less than 900° C., more preferably from greater than 650 to 880° C., more preferably from 700 to 870° C., more preferably from greater than 700 to 860° C., more preferably from 750 to 850° C., more preferably from 750 to less than 850° C., more preferably from 760 to 840° C., more preferably from 770 to 830° C., more preferably from 780 to 820° C., more preferably from 790 to 810° C., and more preferably from 795 to 805° C.; and wherein the atmosphere under which calcining of the zeolitic material in (5) is effected contains less than 10 vol.-% of H₂O, preferably 8 vol.-% or less, more preferably 5 vol.-% or less, more preferably 3 vol.-% or less, more preferably 1 vol.-% or less, more preferably 0.5 vol.-% or less, more preferably 0.1 vol.-% or less, more preferably 0.05 vol.-% or less, more preferably 0.01 vol.-% or less, more preferably 0.005 vol.-% or less, and more preferably 0.001 vol.-% or less of H₂O. 2. The process of embodiment 1, wherein the atmosphere under which calcining of the zeolitic material in (5) is effected contains less than 10 vol.-% of H₂, preferably 8 vol.-% or less, more preferably 5 vol.-% or less, more preferably 3 vol.-% or less, more preferably 1 vol.-% or less, more preferably 0.5 vol.-% or less, more preferably 0.1 vol.-% or less, more preferably 0.05 vol.-% or less, more preferably 0.01 vol.-% or less, more preferably 0.005 vol.-% or less, and more preferably 0.001 vol.-% or less of H₂.
3. The process of embodiment 1 or 2, wherein calcining of the zeolitic material in (3) and/or (5) is effected under air as the atmosphere.
4. The process of any of embodiments 1 to 3, wherein the temperature of calcination in (3) is in the range of from 400 to 850° C., preferably from 450 to 700° C., more preferably from 550 to 650° C., and more preferably from 575 to 625° C.
5. The process of any of embodiments 1 to 4, wherein calcining in (3) and/or (5) is conducted for a period in the range of from 0.5 to 24 h, preferably from 1 to 16 h, more preferably from 2 to 12 h, more preferably from 2.5 to 9 h, more preferably from 3 to 7 h, more preferably from 3.5 to 6.5 h, more preferably from 4 to 6 h, and more preferably from 4.5 to 5.5 h.
6. The process of any of embodiments 1 to 5, wherein in (2) the mixture is heated at a temperature ranging from 90 to 250° C., preferably from 100 to 230° C., more preferably from 110 to 210° C., more preferably from 130 to 190° C., more preferably from 140 to 180° C., more preferably from 150 to 170° C., and more preferably from 155 to 165° C.
7. The process of any of embodiments 1 to 6, wherein the heating in (2) is conducted under autogenous pressure, preferably under solvothermal conditions, more preferably under hydrothermal conditions, wherein preferably heating in (2) is performed in a pressure tight vessel, preferably in an autoclave.
8. The process of any of embodiments 1 to 7, wherein in (2) the mixture is heated for a period ranging from 0.25 to 12 d, preferably from 0.5 to 9 d, more preferably from 1 to 7 d, more preferably from 2 to 6 d, more preferably from 3 to 7 d, more preferably from 2.5 to 5.5 d, more preferably from 3 to 5 d, and more preferably from 3.5 to 4.5 d.
9. The process of any of embodiments 1 to 8, wherein the atmosphere under which calcining of the zeolitic material in (3) is effected contains H₂ in the range of from 1 to 99 vol.-%, preferably from 3 to 90 vol.-%, more preferably from 5 to 70 vol.-%, more preferably from 8 to 50 vol.-%, more preferably from 10 to 40 vol.-%, more preferably from 13 to 30 vol.-% more preferably from 15 to 25 vol.-%, more preferably from 17 to 23 vol.-%, and more preferably from 19 to 21 vol.-%.
10. The process of embodiment 9, wherein the hydrogen gas containing atmosphere further comprises one or more inert gases in addition to hydrogen gas, wherein preferably the hydrogen gas containing atmosphere further comprises one or more inert gases selected from the group consisting of nitrogen, helium, neon, argon, xenon, carbon monoxide, carbon dioxide, and mixtures of two or more thereof, more preferably from the group consisting of nitrogen, argon, carbon monoxide, carbon dioxide, and mixtures of two or more thereof, wherein more preferably the hydrogen gas containing atmosphere further comprises nitrogen and/or argon, and more preferably nitrogen.
11. The process of embodiment 9 or 10, wherein the hydrogen gas containing atmosphere contains 1 vol.-% or less of oxygen gas, preferably 0.5 vol.-% or less, more preferably 0.1 vol.-% or less, more preferably 0.05 vol.-% or less, more preferably 0.01 vol.-% or less, more preferably 0.005 vol.-% or less, more preferably 0.001 vol.-% or less, more preferably 0.0005 vol.-% or less, and more preferably 0.0001 vol.-% or less, wherein more preferably the hydrogen gas containing atmosphere does not contain oxygen gas.
12. The process of any of embodiments 1 to 11, wherein the molar ratio SDA:SiO₂ of the one or more structure directing agents (SDA) to SiO₂ in the framework structure of the first zeolitic material in the mixture prepared according to (1) ranges from 0.01 to 2, preferably from 0.02 to 1.5, more preferably from 0.03 to 1, more preferably from 0.04 to 0.8, more preferably from 0.06 to 0.5, more preferably from 0.08 to 0.3, more preferably from 0.1 to 0.35, more preferably from 0.12 to 0.25, and more preferably from 0.15 to 0.2.
13. The process of any of embodiments 1 to 12, wherein the mixture prepared according to (1) further comprises one or more solvents, wherein said one or more solvents preferably comprises water, preferably distilled water, wherein more preferably water is contained as the one or more solvents in the mixture prepared according to (1), preferably distilled water.
14. The process of embodiment 13, wherein the mixture prepared according to (1) comprises water, wherein the molar ratio H₂O:SiO₂ of water to SiO₂ in the framework structure of the first zeolitic material in the mixture prepared according to (1) ranges from 1 to 80, preferably from 5 to 50, more preferably from 10 to 30, and more preferably from 15 to 20.
15. The process of any of embodiments 1 to 14, wherein after (2) and prior to (3), the process further comprises one or more of:
   (2a) isolating the zeolitic material obtained in (2), preferably by filtration, and/or
   (2b) washing the zeolitic material obtained in (2) or (2a), and/or
   (2c) drying the zeolitic material obtained in any of (2), (2a), or (2b).
16. The process of any of embodiments 1 to 15, wherein X is selected from the group consisting of Al, B, In, Ga, and mixtures of two or more thereof, X preferably being Al and/or B, and more preferably being Al.
17. The process of any of embodiments 1 to 16, wherein the first zeolitic material has a framework structure selected from the group consisting of FAU-, GIS-, MOR-, BEA-, MFI-, and LTA-type framework structures, including mixtures of two or more thereof, preferably from the group consisting of FAU-, MOR-, BEA-, and MFI-type framework structures, more preferably from the group consisting of FAU-, BEA-, and MFI-type framework structures, wherein more preferably the first zeolitic material has an FAU- and/or MFI-type framework structure, wherein more preferably the first zeolitic material has an FAU-type framework structure.
18. The process of any of embodiments 1 to 17, wherein the first zeolitic material having an FAU-type framework structure is selected from the group consisting of ZSM-3, Faujasite, [Al—Ge—O]-FAU, CSZ-1, ECR-30, Zeolite X, Zeolite Y, LZ-210, SAPO-37, ZSM-20, Na-X, US-Y, Na-Y, [Ga—Ge—O]-FAU, Li-LSX, [Ga—Al—Si—O]-FAU, and [Ga—Si—O]-FAU, including mixtures of two or more thereof,
   preferably from the group consisting of ZSM-3, Faujasite, CSZ-1, ECR-30, Zeolite X, Zeolite Y, LZ-210, ZSM-20, Na-X, US-Y, Na-Y, and Li-LSX, including mixtures of two or more thereof,
   more preferably from the group consisting of Faujasite, Zeolite X, Zeolite Y, Na-X, US-Y, and Na-Y, including mixtures of two or more thereof,
   more preferably from the group consisting of Faujasite, Zeolite X, and Zeolite Y, including mixtures of two or more thereof,
   wherein more preferably the first zeolitic material having an FAU-type framework structure comprises zeolite X and/or zeolite Y, preferably zeolite Y,
   wherein more preferably the first zeolitic material having an FAU-type framework structure is zeolite X and/or zeolite Y, preferably zeolite Y.
19. The process of any of embodiments 1 to 18, wherein the first zeolitic material having an MFI-type framework structure is selected from the group consisting of Silicalite, ZSM-5, [Fe—Si—O]-MFI, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, MnS-1, and FeS-1, including mixtures of two or more thereof, preferably from the group consisting of Silicalite, ZSM-5, AMS-1B, AZ-1, Encilite, FZ-1, LZ-105, Mutinaite, NU-4, NU-5, TS-1, TSZ, TSZ-III, TZ-01, USC-4, USI-108, ZBH, ZKQ1B, and ZMQ-TB, including mixtures of two or more thereof,
   wherein more preferably the first zeolitic material having an MFI-type framework structure comprises Silicalite and/or ZSM-5, preferably ZSM-5,
   wherein more preferably the first zeolitic material having an MFI-type framework structure is zeolite Silicalite and/or ZSM-5, preferably ZSM-5.
20. The process of any of embodiments 1 to 19, wherein the first zeolitic material having a BEA-type framework structure is selected from the group consisting of zeolite beta, Tschernichite, [B—Si—O]-*BEA, CIT-6, [Ga—Si—O]-*BEA, Beta polymorph B, SSZ-26, SSZ33, Beta polymorph A, [Ti—Si—O]-*BEA, and pure silica beta, including mixtures of two or more thereof,
   preferably from the group consisting of zeolite beta, CIT-6, Beta polymorph B, SSZ-26, SSZ-33, Beta polymorph A, and pure silica beta, including mixtures of two or more thereof,
   wherein more preferably the first zeolitic material having a BEA-type framework structure comprises zeolite beta, preferably zeolite beta obtained from organotemplate-free synthesis,
   wherein more preferably the first zeolitic material having a BEA-type framework structure is zeolite beta, preferably zeolite beta obtained from organotemplate-free synthesis.
21. The process of any of embodiments 1 to 20, wherein the first zeolitic material having a GIS-type framework structure is selected from the group consisting of zeolite P, TMA-gismondine, Na-P1, Amicite, Gobbinsite, High-silica Na-P, Na-P2, SAPO-43, Gismondine, MAPSO-43, MAPSO-43, Garronite, Synthetic amicite, Synthetic garronite, Synthetic gobbinsite, [Ga—Si—O]-GIS, Synthetic Ca-garronite, Low-silica Na-P (MAP), [Al—Ge—O]-GIS, including mixtures of two or more thereof,
   preferably from the group consisting of zeolite P, TMA-gismondine, Na-P1, Amicite, Gobbinsite, High-silica Na-P, Na-P2, Gismondine, Garronite, Synthetic amicite, Synthetic garronite, Synthetic gobbinsite, [Ga—Si—O]-GIS, Synthetic Ca-garronite, [Al—Ge—O]-GIS, including mixtures of two or more thereof,
   more preferably from the group consisting of zeolite P, TMA-gismondine, Na-P1, Amicite, Gobbinsite, High-silica Na-P, Na-P2, Gismondine, Garronite, Synthetic amicite, Synthetic garronite, Synthetic gobbinsite, Synthetic Ca-garronite, including mixtures of two or more thereof,
   more preferably from the group consisting of zeolite P, Na-P1, High-silica Na-P, Na-P2, including mixtures of two or more thereof,
   wherein more preferably the first zeolitic material having a GIS-type framework structure comprises zeolite P,
   wherein more preferably the first zeolitic material having a GIS-type framework structure is zeolite P.
22. The process of any of embodiments 1 to 21, wherein the first zeolitic material having an MOR-type framework structure is selected from the group consisting of Mordenite, [Ga—SiO—O]-MOR, Maricopaite, Ca-Q, LZ-211, Na-D, RMA-1, including mixtures of two or more thereof,
   wherein preferably the first zeolitic material having an MOR-type framework structure comprises Mordenite,
   wherein more preferably the first zeolitic material having an MOR-type framework structure is Mordenite.
23. The process of any of embodiments 1 to 22, wherein the first zeolitic material having an LTA-type framework structure is selected from the group consisting of Linde Type A (zeolite A), Alpha, [Al—Ge—O]-LTA, N-A, LZ-215, SAPO-42, ZK-4, ZK-21, Dehyd. Linde Type A (dehyd. zeolite A), ZK-22, ITQ-29, UZM-9, including mixtures of two or more thereof, preferably from the group consisting of Linde Type A, Alpha, N-A, LZ-215, SAPO-42, ZK4, ZK-21, Dehyd. Linde Type A, ZK-22, ITQ-29, UZM-9, including mixtures of two or more thereof,
   more preferably from the group consisting of Linde Type A, Alpha, N-A, LZ-215, ZK-4, ZK21, Dehyd. Linde Type A, ZK-22, ITQ-29, UZM-9, including mixtures of two or more thereof,
   more preferably from the group consisting of Linde Type A, Alpha, N-A, LZ-215, ZK-4, ZK21, ZK-22, ITQ-29, UZM-9, including mixtures of two or more thereof.
24. The process of any of embodiments 1 to 23, wherein the first zeolitic material having an FER-type framework structure is selected from the group consisting of Ferrierite, [Ga—SiO]-FER, [Si—O]-FER, FU-9, ISI-6, NU-23, Sr-D, ZSM-35, and [B—Si—O]-FER, including mixtures of two or more thereof,
   preferably from the group consisting of Ferrierite, FU-9, ISI-6, NU-23, and ZSM-35, including mixtures of two or more thereof, wherein more preferably the first zeolitic material having an FER-type framework structure is Ferrierite.
25. The process of any of embodiments 1 to 24, wherein the first zeolitic material having an TON-type framework structure is selected from the group consisting of Theta-1, ZSM-22, ISM, KZ-2, and NU-10, including mixtures of two or more thereof,
   wherein preferably the first zeolitic material having a TON-type framework structure is ZSM-22.
26. The process of any of embodiments 1 to 25, wherein the first zeolitic material having an MTT-type framework structure is selected from the group consisting of ZSM-23, EU-13, ISI-4, and KZ-1, including mixtures of two or more thereof,
   wherein preferably the first zeolitic material having a MTT-type framework structure is ZSM-23.
27. The process of any of embodiments 1 to 26, wherein the second zeolitic material obtained in (2) having an AEI-type framework structure is selected from the group consisting of SSZ-39, SAPO-18, SIZ-8, including mixtures of two or more thereof, wherein more preferably the second zeolitic material obtained in (2) comprises SSZ-39, and wherein more preferably the second zeolitic material obtained in (2) is SSZ-39.
28. The process of any of embodiments 1 to 27, wherein the mixture prepared in (1) and heated in (2) further comprises at least one source for OH⁻, wherein said at least one source for OH⁻ preferably comprises a metal hydroxide, more preferably a hydroxide of an alkali metal M, more preferably sodium and/or potassium hydroxide, and more preferably sodium hydroxide, wherein more preferably the at least one source for OH⁻ is sodium hydroxide.
29. The process of embodiment 28, wherein the OH⁻:SiO₂ molar ratio of OH⁻ to SiO₂ in the framework structure of the first zeolitic material in the mixture prepared according to (1) is in the range of from 0.01 to 1, preferably from 0.03 to 0.7, more preferably from 0.05 to 0.5, more preferably from 0.1 to 0.45, more preferably from 0.15 to 0.4, more preferably from 0.2 to 0.35, and more preferably from 0.25 to 0.3.
30. The process of any of embodiments 1 to 29, wherein the one or more structure directing agents comprises one or more tetraalkylammonium cation R¹R²R³R⁴N⁺-containing compounds, wherein R¹, R², R³ and R⁴ independently from one another stand for alkyl, and wherein R³ and R⁴ form a common alkyl chain.
31. The process of embodiment 30, wherein R¹ and R² independently from one another stand for optionally substituted and/or optionally branched (C₁-C₆)alkyl, preferably (C₁-C₅)alkyl, more preferably (C₁-C₄)alkyl, more preferably (C₁-C₃)alkyl, and more preferably for optionally substituted methyl or ethyl, wherein more preferably R¹ and R² independently from one another stand for optionally substituted methyl or ethyl, preferably unsubstituted methyl or ethyl, wherein more preferably R¹ and R² independently from one another stand for optionally substituted methyl, preferably unsubstituted methyl.
32. The process of embodiment 30 or 31, wherein R³ and R⁴ form a common derivatized or underivatized, preferably underivatized alkyl chain, preferably a common (C₄-C₈)alkyl chain, more preferably a common (C₄-C₇)alkyl chain, more preferably a common (C₄-C₆)alkyl chain, wherein more preferably said common alkyl chain is a derivatized or underivatized, preferably underivatized C4 or C5 alkyl chain, and more preferably a derivatized or underivatized, preferably underivatized C5 alkyl chain.
33. The process of any of embodiments 30 to 32, wherein the one or more tetraalkylammonium cation R¹R²R³R⁴N⁺-containing compounds comprise one or more ammonium compounds selected from the group consisting of derivatized or underivatized, preferably underivatized N,N-di(C₁-C₄)alkyl-3,5-di(C₁-C₄)alkylpyrrolidinium compounds, N,N-di(C₁-C₄)alkyl-3,5-di(C₁-C₄)alkylpiperidinium compounds, N,N-di(C₁-C₄)alkyl-3,5-di(C₁-C₄)alkylhexahydroazepinium compounds, N,N-di(C₁-C₄)alkyl-2,6-di(C₁-C₄)alkylpyrrolidinium compounds, N,N-di(C₁-C₄)alkyl-2,6-di(C₁-C₄)alkylpiperidinium compounds, N,N-di(C₁-C₄)alkyl-2,6-di(C₁-C₄)alkylhexahydroazepinium compounds, and mixtures of two or more thereof,
   preferably from the group consisting of N,N-di(C₁-C₄)alkyl-3,5-di(C₁-C₄)alkylpyrrolidinium compounds, N,N-di(C₁-C₄)alkyl-3,5-di(C₁-C₄)alkylpiperidinium compounds, N,N-di(C₁-C₄)alkyl-3,5-di(C₁-C₄)alkylhexahydroazepinium compounds, N,N-di(C₁-C₄)alkyl-2,6-di(C₁-C₄)alkylpyrrolidinium compounds, N,N-di(C₁-C₄)alkyl-2,6-di(C₁-C₄)alkylpiperidinium compounds, N,N-di(C₁-C₄)alkyl-2,6-di(C₁-C₄)alkylhexahydroazepinium compounds, and mixtures of two or more thereof,
   more preferably from the group consisting of N,N-di(C₁-C₃)alkyl-3,5-di(C₁-C₃)alkylpyrrolidinium compounds, N,N-di(C₁-C₃)alkyl-3,5-di(C₁-C₃)alkylpiperidinium compounds, N,N-di(C₁-C₃)alkyl-3,5-di(C₁-C₃)alkylhexahydroazepinium compounds, N,N-di(C₁-C₃)alkyl-2,6-di(C₁-C₃)alkylpyrrolidinium compounds, N,N-di(C₁-C₃)alkyl-2,6-di(C₁-C₃)alkylpiperidinium compounds, N,N-di(C₁-C₃)alkyl-2,6-di(C₁-C₃)alkylhexahydroazepinium compounds, and mixtures of two or more thereof,
   more preferably from the group consisting of N,N-di(C₁-C₂)alkyl-3,5-di(C₁-C₂)alkylpyrrolidinium compounds, N,N-di(C₁-C₂)alkyl-3,5-di(C₁-C₂)alkylpiperidinium compounds, N,N-di(C₁-C₂)alkyl-3,5-di(C₁-C₂)alkylhexahydroazepinium compounds, N,N-di(C₁-C₂)alkyl-2,6-di(C₁-C₂)alkylpyrrolidinium compounds, N,N-di(C₁-C₂)alkyl-2,6-di(C₁-C₂)alkylpiperidinium compounds, N,N-di(C₁-C₂)alkyl-2,6-di(C₁-C₂)alkylhexahydroazepinium compounds, and mixtures of two or more thereof,
   more preferably from the group consisting of N,N-di(C₁-C₂)alkyl-3,5-di(C₁-C₂)alkylpiperidinium compounds, N,N-di(C₁-C₂)alkyl-2,6-di(C₁-C₂)alkylpiperidinium compounds, and mixtures of two or more thereof, wherein more preferably the one or more tetraalkylammonium cation R¹R²R³R⁴N⁺-containing compounds comprise one or more N,N-dimethyl-3,5-dimethylpiperidinium and/or N,N-dimethyl-2,6-dimethylpiperidinium compounds, preferably one or more N,N-dimethyl-3,5-dimethylpiperidinium compounds.
34. The process of any of embodiments 30 to 33, wherein the one or more tetraalkylammonium cation R¹R²R³R⁴N⁺-containing compounds are salts, preferably one or more salts selected from the group consisting of halides, sulfate, nitrate, phosphate, acetate, and mixtures of two or more thereof, more preferably from the group consisting of bromide, chloride, hydroxide, sulfate, and mixtures of two or more thereof, wherein more preferably the one or more tetraalkylammonium cation R¹R²R³R⁴N⁺-containing compounds are tetraalkylammonium hydroxides and/or bromides, and more preferably tetraalkylammonium hydroxides.
35. The process of any of embodiments 30 to 34, wherein the mixture prepared according to (1) further comprises distilled water, wherein the molar ratio H₂O:SiO₂ of water to SiO₂ in the framework structure of the first zeolitic material in the mixture prepared according to (1) ranges from 1 to 80, preferably from 5 to 60, more preferably from 10 to 50, more preferably from 15 to 45, more preferably from 20 to 40, more preferably from 25 to 35, and more preferably from 28 to 32.
36. The process of any of embodiments 30 to 35, wherein the molar ratio R¹R²R³R⁴N⁺: SiO₂ of the one or more tetraalkylammonium cations to SiO₂ in the framework structure of the first zeolitic material in the mixture prepared according to (1) ranges from 0.01 to 1.5, preferably from 0.05 to 1, more preferably from 0.1 to 0.8, more preferably from 0.3 to 0.5, more preferably from 0.5 to 0.3, more preferably from 0.8 to 0.25, more preferably from 0.1 to 0.2, more preferably from 0.12 to 0.18, and more preferably from 0.14 to 0.16.
37. The process of any of embodiments 30 to 36, wherein the framework structure of the first zeolitic material displays an SiO₂:X₂O₃ molar ratio ranging from 1 to 50, preferably from 2 to 25, more preferably from 3.5 to 15, more preferably from 3 to 10, more preferably from 4.5 to 8, and more preferably from 5 to 6.
38. The process of any of embodiments 30 to 37, wherein the mixture prepared in (1) and heated in (2) further comprises at least one source for OH⁻, wherein the OH⁻:SiO₂ molar ratio of OH to SiO₂ in the framework structure of the first zeolitic material in the mixture prepared according to (1) is in the range of from 0.1 to 1, preferably from 0.3 to 0.7, more preferably from 0.4 to 0.5, and more preferably from 0.43 to 0.48.
39. The process of any of embodiments 1 to 29, wherein the one or more structure directing agents comprises one or more quaternary phosphonium cation R¹R²R³R⁴P⁺-containing compounds, wherein R¹, R², R³, and R⁴ independently from one another stand for optionally substituted and/or optionally branched (C₁-C₆)alkyl, preferably (C₁-C₅)alkyl, more preferably (C₁-C₄)alkyl, more preferably (C₂-C₃)alkyl, and more preferably for optionally substituted methyl or ethyl, wherein more preferably R¹, R², R³, and R⁴ stand for optionally substituted ethyl, preferably unsubstituted ethyl.
40. The process of embodiment 39, wherein the one or more quaternary phosphonium cation R¹R²R³R⁴P⁺-containing compounds are salts, preferably one or more salts selected from the group consisting of halides, preferably chloride and/or bromide, more preferably chloride, hydroxide, sulfate, nitrate, phosphate, acetate, and mixtures of two or more thereof, more preferably from the group consisting of chloride, hydroxide, sulfate, and mixtures of two or more thereof, wherein more preferably the one or more quaternary phosphonium cation containing compounds are hydroxides and/or chlorides, and more preferably hydroxides.
41. The process of embodiment 39 or 40, wherein the mixture prepared according to (1) further comprises distilled water, wherein the molar ratio H₂O:SiO₂ of water to SiO₂ in the framework structure of the first zeolitic material in the mixture prepared according to (1) ranges from 1 to 80, preferably from 1.5 to 50, more preferably from 2 to 30, more preferably from 2.5 to 15, more preferably from 3 to 10, more preferably from 3.5 to 8, more preferably from 4 to 6, and more preferably from 4.5 to 5.5.
42. The process of any of embodiments 39 to 41, wherein the molar ratio R¹R²R³R⁴P⁺: SiO₂ of the one or more quaternary phosphonium cations to SiO₂ in the framework structure of the first zeolitic material in the mixture prepared according to (1) ranges from 0.01 to 2, preferably from 0.05 to 1.5, more preferably from 0.1 to 1, more preferably from 0.3 to 0.8, more preferably from 0.5 to 0.5, more preferably from 0.8 to 0.4, more preferably from 0.1 to 0.35, more preferably from 0.12 to 0.3, more preferably from 0.15 to 0.25, more preferably from 0.17 to 0.23, and more preferably from 0.19 to 0.21.
43. The process of any of embodiments 39 to 42, wherein the framework structure of the first zeolitic material displays an SiO₂:X₂O₃ molar ratio ranging from 1 to 150, preferably from 5 to 100, more preferably from 10 to 70, more preferably from 15 to 50, more preferably from 20 to 40, more preferably from 25 to 35, and more preferably from 28 to 32.
44. The process of any of embodiments 39 to 43, wherein the mixture prepared in (1) and heated in (2) further comprises at least one source for OH⁻, wherein the OH⁻:SiO₂ molar ratio of OH to SiO₂ in the framework structure of the first zeolitic material in the mixture prepared according to (1) is in the range of from 0.01 to 0.3, preferably from 0.03 to 0.2, more preferably from 0.05 to 0.15, and more preferably from 0.08 to 0.12.
45. A zeolitic material having an AEI-type framework structure obtainable and/or obtained according to the process of any of embodiments 1 to 44.
46. A zeolitic material having an AEI-type framework structure comprising SiO₂ and X₂O₃ in its framework structure, preferably obtainable and/or obtained according to the process of any of embodiments 1 to 44, wherein X stands for a trivalent element, and wherein the deconvoluted ammonia temperature programmed desorption spectrum of the zeolitic material displays a first peak (peak I) in the range of from 205 to 270° C. and a second peak (peak II) in the range of from 300 to 460° C., wherein the integration of peak I affords an amount of acid sites in the range of from 0.07 to 0.35 mmol/g, and the integration of peak II affords an amount of acid sites in the range of from 0.25 to 0.4 mmol/g.
47. The zeolitic material of embodiment 46, wherein peak I is in the range of from 208 to 260° C., preferably from 210 to 240° C., more preferably from 212 to 235° C., more preferably from 213 to 230° C., more preferably from 214 to 225° C., more preferably from 215 to 220° C., and more preferably from 216 to 218° C., wherein more preferably peak I is at 217° C.
48. The zeolitic material of embodiment 46 or 47, wherein the integration of peak I affords an amount of acid sites in the range of from 0.09 to 0.3 mmol/g, preferably from 0.11 to 0.25 mmol/g, more preferably from 0.12 to 0.2 mmol/g, more preferably from 0.125 to 0.17 mmol/g, and more preferably from 0.13 to 0.15 mmol/g.
49. The zeolitic material of any of embodiments 46 to 48, wherein peak II is in the range of from 310 to 430° C., preferably from 315 to 400° C., more preferably from 320 to 380° C., more preferably from 325 to 360° C., more preferably from 330 to 350° C., more preferably from 333 to 345° C., and more preferably from 335 to 340° C.
50. The zeolitic material of any of embodiments 46 to 49, wherein the integration of peak II affords an amount of acid sites in the range of from 0.28 to 0.37 mmol/g, preferably from 0.3 to 0.35 mmol/g, more preferably from 0.31 to 0.34 mmol/g, and more preferably from 0.32 to 0.33 mmol/g.
51. The zeolitic material of any of embodiments 46 to 50, wherein the ratio of the amount of acid sites from the integration of peak I to the amount of acid sites from the integration of peak II is in the range of from 0.35 to 0.7, preferably from 0.38 to 0.6, more preferably from 0.4 to 0.5, more preferably from 0.41 to 0.47, more preferably from 0.42 to 0.45, and more preferably from 0.43 to 0.44.
52. The zeolitic material of any of embodiments 46 to 51, wherein the deconvoluted ammonia temperature programmed desorption spectrum of the zeolitic material further displays a third peak (peak III) in the range of from 160 to 177° C., preferably from 163 to 174° C., more preferably from 165 to 172° C., more preferably from 166 to 171° C., more preferably from 167 to 170° C., and more preferably from 168 to 169° C.
53. The zeolitic material of embodiment 52, wherein the integration of peak III affords an amount of acid sites in the range of from 0.07 to 0.3 mmol/g, preferably from 0.09 to 0.25 mmol/g, more preferably from 0.1 to 0.2 mmol/g, more preferably from 0.11 to 0.17 mmol/g, more preferably from 0.11 to 0.15 mmol/g, more preferably from 0.12 to 0.14 mmol/g, and more preferably from 0.12 to 0.13 mmol/g.
54. The zeolitic material of any of embodiments 46 to 53, wherein the CO-FTIR spectrum of the zeolitic material displays a first peak in the range of from 3290 to 3315 cm⁻¹, preferably from 3295 to 3310 cm⁻¹, more preferably from 3300 to 3306 cm⁻¹, more preferably from 3301 to 3305 cm⁻¹, and more preferably from 3302 to 3304 cm⁻¹, and a second peak in the range of from 3420 to 3470 cm⁻¹, more preferably from 3425 to 3465 cm⁻¹, more preferably from 3430 to 3460 cm⁻¹, more preferably from 3435 to 3456 cm⁻¹, more preferably from 3437 to 3453 cm⁻¹, and more preferably from 3439 to 3451 cm⁻¹, wherein the maximum absorbance of the second peak is equal to or greater than the maximum absorbance of the first peak, wherein preferably the maximum absorbance of the second peak is greater than the maximum absorbance of the first peak.
55. The zeolitic material of any of embodiments 46 to 54, wherein the SiO₂:X₂O₃ molar ratio of SiO₂ to X₂O₃ respectively in the framework structure of the zeolitic material is in the range of from 2 to 150, preferably of from 4 to 100, more preferably of from 8 to 50, more preferably of from 12 to 35, more preferably of from 16 to 30, more preferably of from 18 to 26, and more preferably of from 20 to 24.
56. The zeolitic material of any of embodiments 46 to 55, wherein X is selected from the group consisting of Al, B, In, Ga, and mixtures of two or more thereof, X preferably being Al and/or B, and more preferably being Al.
57. The zeolitic material of any of embodiments 46 to 56, wherein the BET surface area of the zeolitic material is in the range of from 400 to 800 m²/g, preferably of from 450 to 750 m²/g, more preferably of from 500 to 700 m²/g, more preferably of from 550 to 680 m²/g, more preferably of from 600 to 670 m²/g, and more preferably of from 630 to 660 m²/g, wherein the BET surface area of the zeolitic material is preferably determined according to ISO 9277:2010.
58. The zeolitic material of any of embodiments 46 to 57, wherein the micropore volume of the zeolitic material is in the range of from 0.1 to 0.3 cm³/g, preferably of from 0.13 to 0.26 cm³/g, more preferably of from 0.15 to 0.24 cm³/g, more preferably of from 0.17 to 0.22 cm³/g, and more preferably of from 0.19 to 0.21 cm³/g, wherein the micropore volume of the zeolitic material is preferably determined according to DIN 66135-3:2001-06.
59. The zeolitic material of any of embodiments 46 to 58, wherein the total pore volume of the zeolitic material is in the range of from 0.35 to 0.55 cm³/g, preferably of from 0.38 to 0.48 cm³/g, more preferably of from 0.4 to 0.45 cm³/g, and more preferably of from 0.41 to 0.42 cm³/g, wherein the total pore volume of the zeolitic material is preferably determined according to ISO 9277:2010.
60. The zeolitic material of any of embodiments 46 to 59, wherein the zeolitic material having an AEI-type framework structure is selected from the group consisting of SSZ-39, SAPO-18, and SIZ-8, including mixtures of two or more thereof, wherein more preferably the zeolitic material comprises SSZ-39, and wherein more preferably the zeolitic material is SSZ39.
61. Process for the conversion of oxygenates to olefins comprising
   (I) providing a gas stream comprising one or more oxygenates;
   (II) contacting the gas stream with a catalyst comprising a zeolitic material according to any of embodiments 45 to 60.
62. The process of embodiment 61, wherein the gas stream provided in step (I) contains one or more oxygenates selected from the group consisting of aliphatic alcohols, ethers, carbonyl compounds, and mixtures of two or more thereof, preferably from the group consisting of C₁-C₆-alcohols, di-C₁-C₃-alkyl ethers, C₁-C₆-aldehydes, C₂-C₆-ketones, and mixtures of two or more thereof, more preferably from the group consisting of C₁-C₄-alcohols, di-C₁-C₂-alkyl ethers, C₁-C₄-aldehydes, C₂-C₄-ketones, and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone, and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone, and mixtures of two or more thereof, more preferably from the group consisting of methanol, ethanol, dimethyl ether, diethyl ether, ethyl methyl ether, and mixtures of two or more thereof, wherein more preferably the gas stream provided in step (I) comprises methanol and/or dimethyl ether, preferably methanol.
63. The process of embodiment 61 or 62, wherein the gas stream provided in step (I) contains the one or more oxygenates in an amount in the range of from 30 to 100 vol.-% of based on the total volume of the gas stream, preferably from 30 to 99.9 vol.-%, more preferably from 30 to 99 vol.-%, more preferably from 30 to 95 vol.-%, more preferably from 30 to 90 vol.-%, more preferably from 30 to 80 vol.-%, more preferably from 30 to 70 vol.-%, more preferably from 30 to 60 vol.-%, more preferably from 30 to 50 vol.-%, and more preferably from 30 to 45 vol.-%.
64. The process of any of embodiments 61 to 63, wherein the gas stream provided in step (I) contains 60 vol.-% or less of H₂O based on the total volume of the gas stream, wherein preferably the gas stream provided in step (I) contains H₂O in the range of from 5 to 60 vol.-%, more preferably from 10 to 55 vol.-%, more preferably from 20 to 50 vol.-%, and more preferably from 30 to 45 vol.-%.
65. The process of any of embodiments 1 to 64, wherein the gas stream provided in step (I) contains 5 vol.-% or less of H₂O based on the total volume of the gas stream, preferably 3 vol.-% or less, more preferably 1 vol.-% or less, more preferably 0.5 vol.-% or less, more preferably 0.1 vol.-% or less, more preferably 0.05 vol.-% or less, more preferably 0.01 vol.-% or less, more preferably 0.005 vol.-% or less, and more preferably 0.001 vol.-% or less.
66. The process of any of embodiments 61 to 65, wherein contacting of the gas stream with the catalyst in step (II) is performed at a temperature in the range of from 200 to 700° C., preferably from 250 to 650° C., more preferably from 300 to 600° C., more preferably from 350 to 560° C., more preferably from 400 to 540° C., more preferably from 430 to 520° C., and more preferably from 450 to 500° C.
67. The process of any of embodiments 61 to 66, wherein contacting of the gas stream with the catalyst in step (II) is performed at a pressure in the range of from 0.1 to 10 bar, preferably from 0.3 to 7 bar, more preferably from 0.5 to 5 bar, more preferably from 0.7 to 3 bar, more preferably from 0.8 to 2.5 bar, more preferably from 0.9 to 2.2 bar, and more preferably from 1 to 2 bar.
68. The process of any of embodiments 61 to 67, wherein the process is performed as a batch process or in a continuous mode, wherein preferably the process is performed at least in part in a continuous mode, wherein more preferably the process is performed in a continuous mode.
69. The process of embodiment 68, wherein the process is performed in a continuous mode, and wherein the weight hourly space velocity (WHSV) of the gas stream in step (II) is in the range of from 0.5 to 50 h⁻¹, preferably from 1 to 30 h⁻¹, more preferably from 2 to 20 h⁻¹, more preferably from 3 to 15 h⁻¹, more preferably from 4 to 10 h⁻¹, and more preferably from 5 to 7 h⁻¹.
70. Use of a zeolitic material of any of embodiments 45 to 60 as a molecular sieve, catalyst, catalyst support, and/or as an adsorbent, preferably as a catalyst and/or as a catalyst support for the selective catalytic reduction (SCR) of nitrogen oxides NO_{R}; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, more preferably as a catalyst and/or as a catalyst support in the conversion of alcohols to olefins, and more preferably as a catalyst for the conversion of alcohols to olefins, preferably of methanol to olefins.

### DESCRIPTION OF THE FIGURES

FIG. 1 shows the results from nitrogen adsorption/desorption measurements for determination of BET surface area and micropore volume performed on the materials of Examples 1 to 4 and Comparative Examples 1 and 2. In the figure, the Si/AI molar ratio is indicated as obtained from ICP-AES, the BET surface area, the total pore volume at P/P₀=0.99, and the micropore volume as obtained by the t-plot method are displayed for the respective materials.

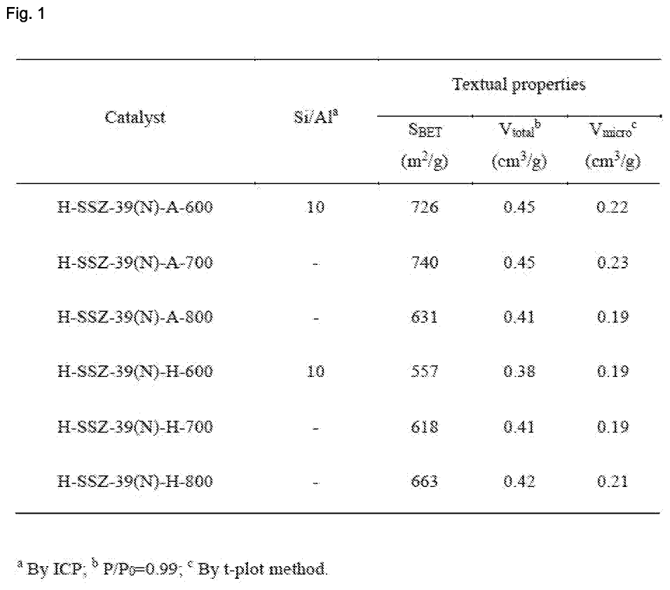

FIG. 2 shows the results from nitrogen adsorption/desorption measurements for determination of BET surface area and micropore volume performed on the materials of Examples 5 to 8 and Comparative Examples 3 and 4. In the figure, the Si/AI molar ratio is indicated as obtained from ICP-AES, the BET surface area, the total pore volume at P/P₀=0.99, and the micropore volume as obtained by the t-plot method are displayed for the respective materials.

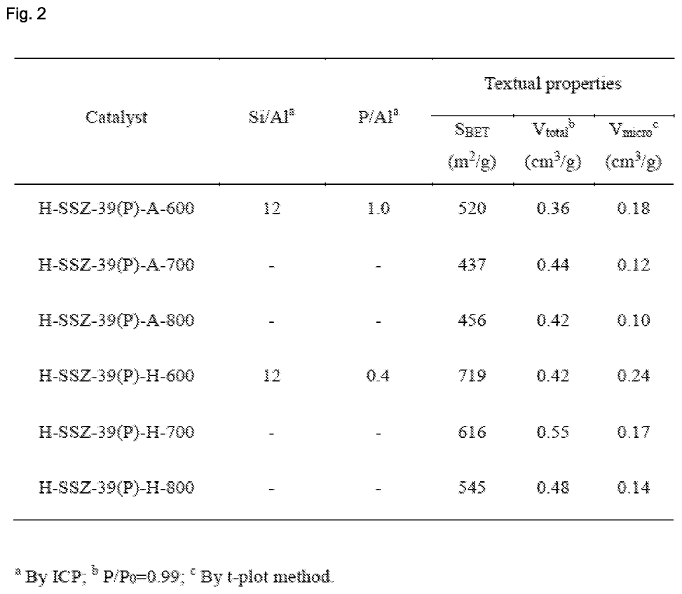

FIG. 3 shows the results from CO-FTIR measurements performed on the materials from Comparative Example 1, Example 1, and Example 2, respectively. In the figure, the absorbance in arbitrary units is displayed along the ordinate and the wavenumber in cm⁻¹ is displayed along the abscissa.

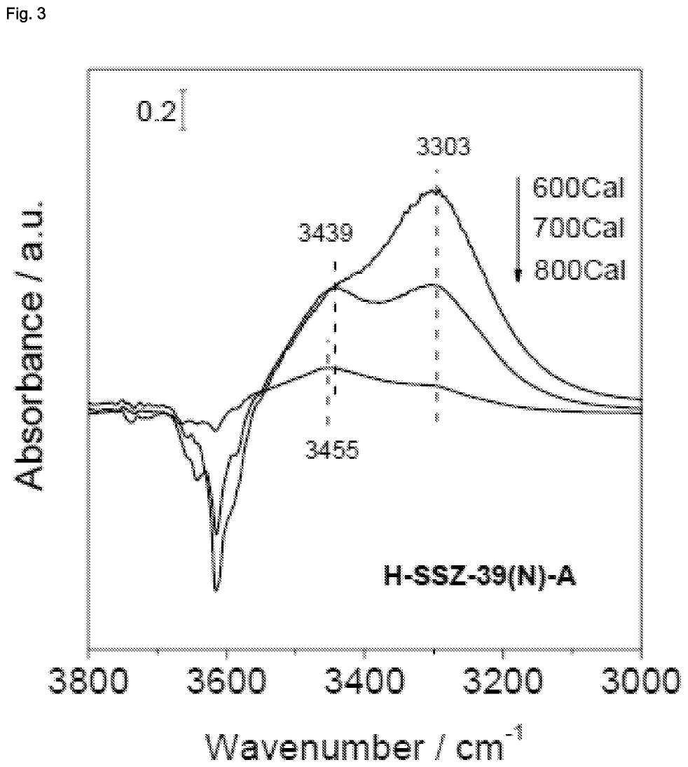

FIG. 4 shows the results from CO-FTIR measurements performed on the materials from Comparative Example 2, Example 3, and Example 4, respectively. In the figure, the absorbance in arbitrary units is displayed along the ordinate and the wavenumber in cm⁻¹ is displayed along the abscissa.

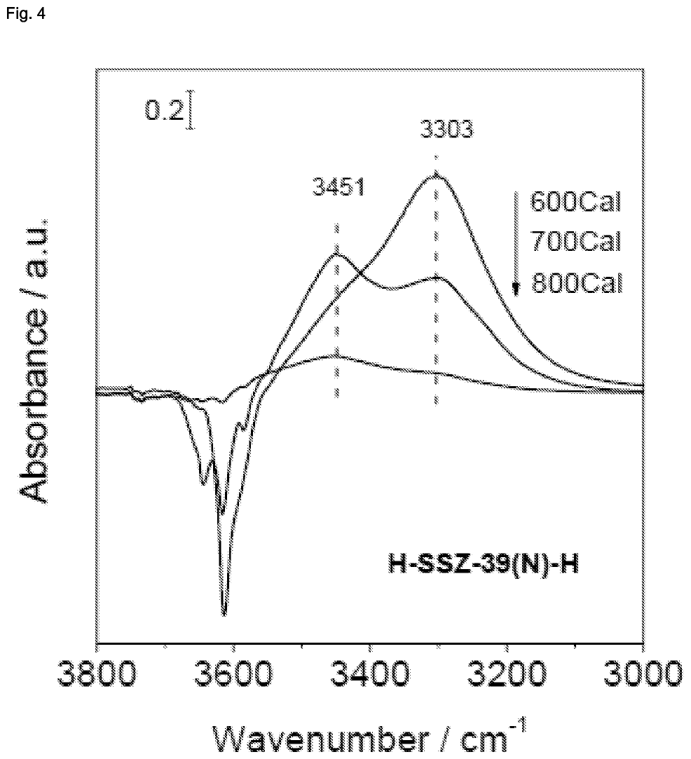

FIG. 5 shows the results from CO-FTIR measurements performed on the materials from Comparative Example 3, Example 5, and Example 6, respectively. In the figure, the absorbance in arbitrary units is displayed along the ordinate and the wavenumber in cm⁻¹ is displayed along the abscissa.

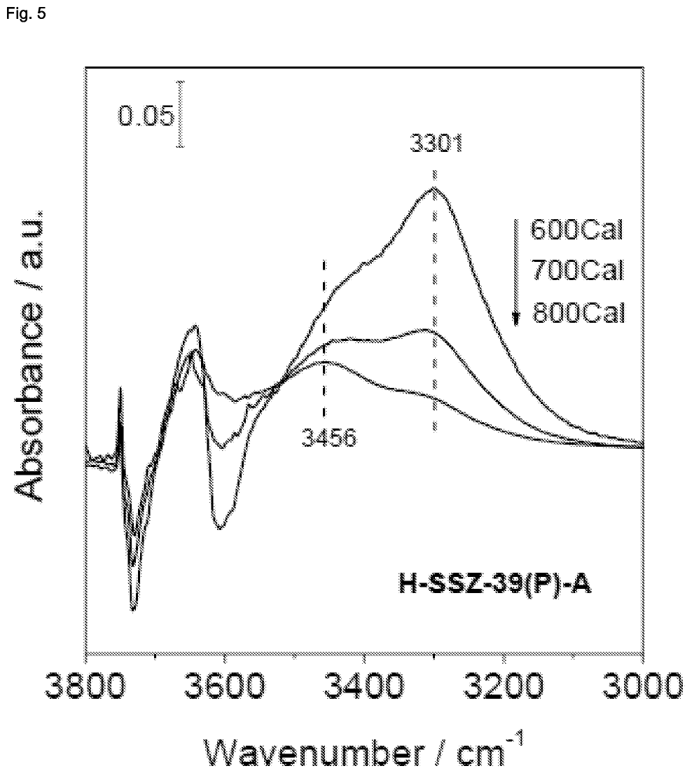

FIG. 6 shows the results from CO-FTIR measurements performed on the materials from Comparative Example 4, Example 7, and Example 8, respectively. In the figure, the absorbance in arbitrary units is displayed along the ordinate and the wavenumber in cm⁻¹ is displayed along the abscissa.

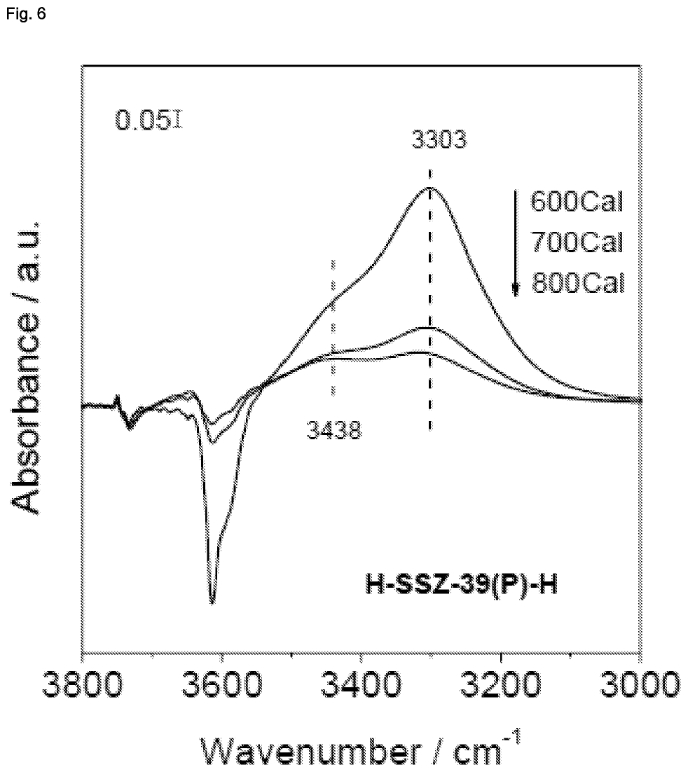

FIG. 7 displays the results from catalytic testing in Example 9 using the catalysts SSZ39(N)-A-600 (Comp. Example 1), SSZ-39(N)-A-700 (Example 1), and SSZ-39(N)A-800 (Example 2). In the figure, the conversion and selectivities in % are displayed along the ordinate and the time on stream in hours is displayed along the abscissa, wherein the conversion of methanol is indicated by the symbol “0”, the selectivity in ethylene by “●”, in propylene by “▪”, in butene by “▴”, in C1-C4 alkanes by “ [symbol] ”, in alkanes of C5 or more by “ [symbol] ”, and in dimethylether by “★”.

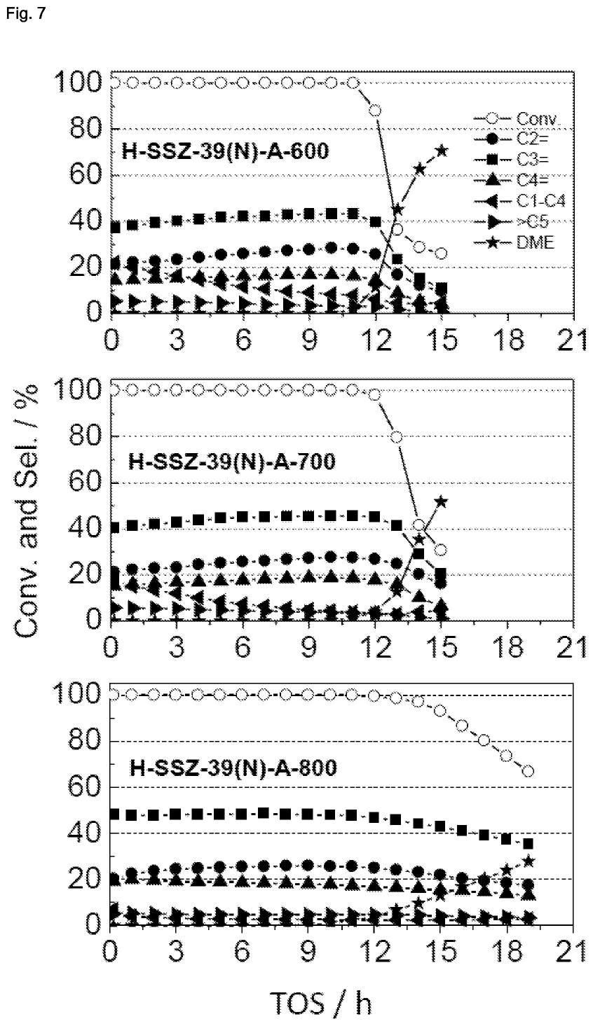

FIG. 8 displays the results from catalytic testing in Example 9 using the SSZ-39(N)-H-600 (Comp. Example 2), SSZ-39(N)-H-700 (Example 3), and SSZ-39(N)-H-800 (Example 4). In the figure, the conversion and selectivities in % are displayed as in FIG. 7.

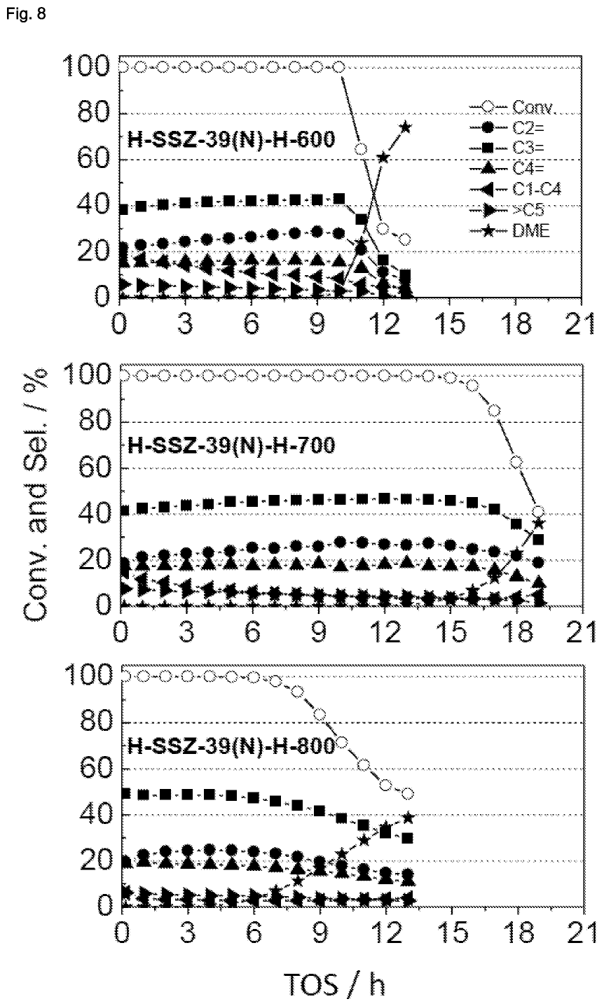

FIG. 9 displays the results from catalytic testing in Example 9 using the catalysts SSZ39(P)-A-600 (Comp. Example 3), SSZ-39(P)-A-700 (Example 5), and SSZ-39(P)A-800 (Example 6). In the figure, the conversion and selectivities in % are displayed as in FIG. 7.

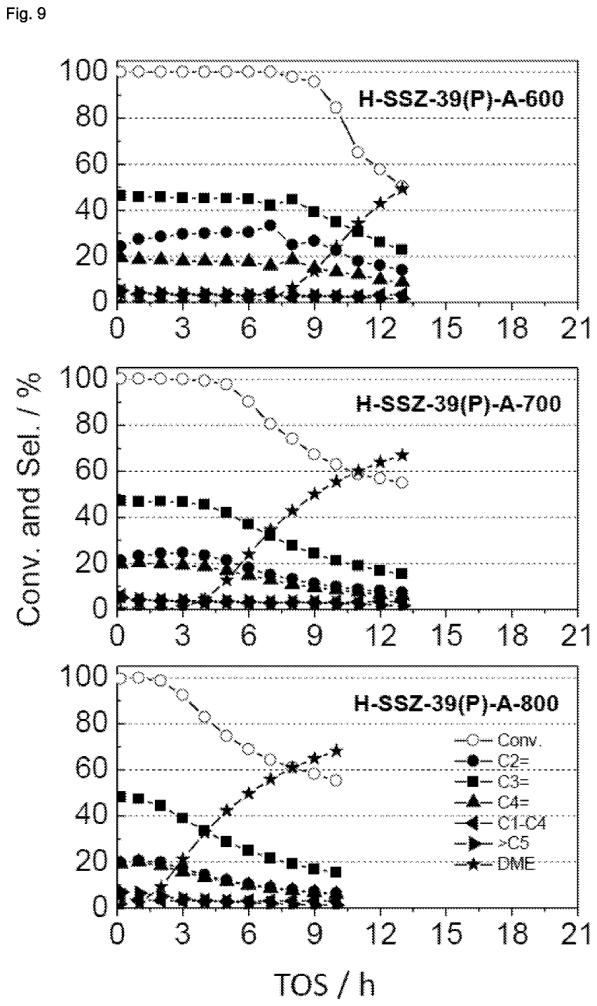

FIG. 10 displays the results from catalytic testing in Example 9 using the catalysts SSZ39(P)-H-600 (Comp. Example 4), SSZ-39(P)-H-700 (Example 7), and SSZ-39(P)H-800 (Example 8). In the figure, the conversion and selectivities in % are displayed as in FIG. 7.

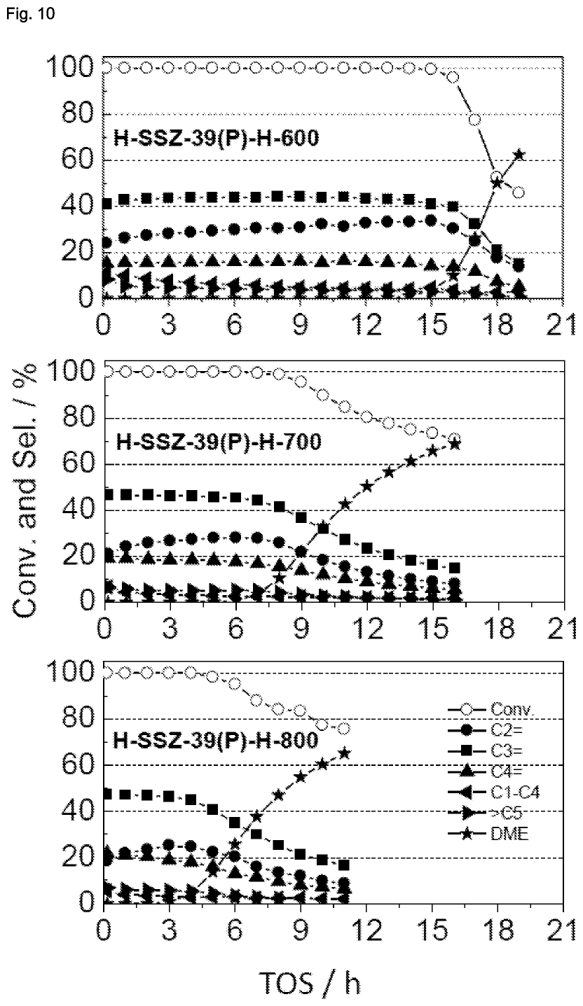

### EXAMPLES

Characterization of the Samples

Elemental analyses were performed on an inductively coupled plasma-atomic emission spectrometer (ICP-AES, Shimadzu ICPE-9000).

Nitrogen Adsorption/Desorption Measurements for Determination of BET Surface Area and Micropore Volume

Nitrogen adsorption/desorption measurements were performed on a Belsorp-mini II analyzer (BEL Japan). Prior to the measurements, all samples were degassed at 350° C. for 3 h. The BET surface area was calculated in the P/P₀ range of 0.01-0.1. The micropore volume was calculated by t-plot method.

NH₃-TPD Method and Data Interpretation: Calculation of Acid Sites and the Type of the Acid Sites

Temperature-programmed desorption of ammonia (NH₃-TPD) profiles were recorded on a BELCAT equipment (BEL Japan). Typically, 25 mg catalyst were pretreated at 600° C. in a He flow (50 mL/min) for 1 h and then cooled to 100° C. Prior to the adsorption of NH₃, the sample was evacuated at 100° C. for 1 h. Approximately 2500 Pa of NH₃ were allowed to make contact with the sample at 100° C. for 30 min. Subsequently, the sample was evacuated to remove weakly adsorbed NH₃ at the same temperature for 30 min. Finally, the sample was heated from 100 to 600° C. at a ramping rate of 10° C./min in a He flow (50 mL/min). A thermal conductivity detector (TCD) was used to monitor desorbed NH₃.

The acid amount calculated according to the deconvolution results form NH₃-TPD profiles and the peak-maximum-temperature listed in Tables 3 and 4 below. Peak III corresponds to NH₃ adsorbed on the non-acidic OH groups and NH₄⁺ by hydrogen bonding. Peaks I and II correspond to NH₃ adsorbed on the true acid sites including Brønsted and Lewis acid sites. The acid strength can be estimated by the position of the peak (i.e., peak-maximum-temperature).

CO FT-IR Measurements: Description of the Measurements Conditions and Analysis of the Type and Amount of Acid Sites

FTIR spectra were obtained by using a Jasco FTIR 4100 spectrometer equipped with a TGS detector at a 4 cm⁻¹ resolution; 64 scans were collected for each spectrum. The powdered samples (˜30 mg) were pelletized into a self-supporting disk of 1 cm in diameter, which was held in a glass cell. After evacuation at 500° C. for 1 h, the sample was cooled back to −120° C. prior to background spectra acquisition. Then CO was introduced into the cell in a pulse mode fashion (˜5 Pa for the first pulse, until total pressure in the IR cell reached ˜1000 Pa). After equilibrium pressure was reached after each pulse, an IR spectrum was acquired. The IR spectra resulting from the subtraction of the background spectra from those with NO adsorbed are shown unless otherwise noted.

The Brønsted acid amount with different strength can be compared for different AEI samples, based on the intensities of bands at ˜3303 and ˜3450 cm⁻¹ related to the strong and medium acid sites, respectively.

### Comparative Example 1: Synthesis of SSZ-39(N)-A-600 Using a Quaternary Ammonium Containing Structure Directing Agent and Calcination Thereof in Air at 600° C.

The following synthesis of SSZ-39(N) is based on the synthetic methodologies described in U.S. Pat. No. 5,958,370 and M. Moliner et al. in Chem. Commun. 2012, 48, pages 8264-8266.

### Synthesis of N,N-dimethyl-3,5-dimethylpiperidinium hydroxide (DMPOH)

First, 24 g of 3,5-dimethylpiperidine (TCI, 98%, cis-trans mixture) were mixed with 220 ml of methanol (Wako, 99.9%) and 42 g of potassium carbonate (Wako, 99.5%). Then, 121 g of methyl iodide (Wako, 99.5%) were added dropwise, and the resultant mixture maintained under reflux for 1 day. After evaporation to partially remove the methanol, chloroform was added and stirred, followed by filtration to remove potassium carbonate. This step was repeated to completely remove the methanol and potassium carbonate. Then, ethanol was added for recrystallization, and diethylether was added to precipitate the iodide salt. After filtration, the solid product was dried and mixed with hydroxide ion exchange resin (DIAION SA10AOH, Mitsubishi) and distilled water. After 1 day, the resin was removed by filtration and the DMPOH aqueous solution with density of 1.051 g mL⁻¹ and molar concentration of 1.817 M was obtained.

### Synthesis of SSZ-39(N)

First, 12.85 of DMPOH aqueous solution were mixed with 10.99 g of 8 M NaOH aqueous solution (Wako) and 62.42 g of distilled water. Then, 1.33 g of Y zeolite (JRC-HY-5.5, Si/Al₂=5.5, JGC Catalysts and Chemicals) were added to the above solution, with stirring for 1 h. Then, 7.91 g fumed silica (Cab-O-SilM5, Cabot) were added to the mixture and stirred for 1 h. The molar composition of the resultant gel was 1 SiO₂:0.05 Al:0.15 DMPOH:0.45 Na:30 H₂O. The thus prepared mother gel was crystallized in an autoclave at 150° C. for 3 days under tumbling condition (30 r.p.m.). The solid crystalline product, a zeolitic material having framework type AEI, was recovered by filtration, washed with distilled water, and dried overnight at 100° C. under air. The thus obtained product displayed an SiO₂: Al₂O₃ molar ratio of 20 as determined from elemental analysis by ICP. The thus obtained SSZ-39(N) product was then calcined in air (“A”) in a muffle furnace at 600° C. for 6 hours which provided the Na-SSZ-39(N)-A. Subsequently, the Na-SSZ-39(N)-A was then NH₄⁺ ion exchanged using 2.5 molar aqueous solution of NH₄NO₃, wherein the weight ratio of the ammonium nitrate solution:zeolite was 100:1, and the resulting mixture was heated to 80° C. for 3 hours, followed by filtration of the solid. The procedure was repeated once to provide NH₄⁺-SSZ-39(N)-A. The thus obtained NH₄⁺-SSZ-39(N)-A was then calcined in air in a muffle furnace at 600° C. for 5 hours which provided the H-form, HSSZ-39(N)-A-600.

### Example 1: Synthesis of SSZ-39(N)-A-700 Using Quaternary Ammonium Containing Structure Directing Agent and Calcination Thereof after Ammonium Ion Exchange at 700° C.

The method of Comparative Example 1 was repeated, wherein the ion exchanged product NH₄⁺-SSZ-39(N)-A was calcined in air in a muffle furnace at 700° C. for 5 hours which provided the H-form, H-SSZ-39(N)-A-700.

### Example 2: Synthesis of SSZ-39(N)-A-800 Using Quaternary Ammonium Containing Structure Directing Agent and Calcination Thereof after Ammonium Ion Exchange at 800° C.

The method of Comparative Example 1 was repeated, wherein the ion exchanged product NH₄⁺-SSZ-39(N)-A was calcined in air in a muffle furnace at 800° C. for 5 hours which provided the H-form, H-SSZ-39(N)-A-800.

### Comparative Example 2: Synthesis of SSZ-39(N)-H-600 Using a Quaternary Ammonium Containing Structure Directing Agent and Calcination Thereof in a Hydrogen-Atmosphere at 600° C.

The method of Comparative Example 1 was repeated, wherein the SSZ-39(N) product was calcined in a flow of hydrogen/nitrogen (H₂: 15 mL/min, N₂: 60 mL/min) (“H”) in a muffle furnace at 600° C. for 6 hours which provided the Na-SSZ-39(N)-H.

As in Comparative Example 1, the Na-SSZ-39(N)-H was then NH₄⁺ ion exchanged as described in Reference Example 1 to provide NH₄⁺-SSZ-39(N)-H, which was then calcined in air at 600° C. for 5 hours which provided the H-form, H-SSZ-39(N)-H-600.

### Example 3: Synthesis of SSZ-39(N)-H-700 Using Quaternary Ammonium Containing Structure Directing Agent and Calcination Thereof after Ammonium Ion Exchange at 700° C.

The method of Comparative Example 2 was repeated, wherein the ion exchanged product NH₄⁺-SSZ-39(N)-H was calcined in air in a muffle furnace at 700° C. for 5 hours which provided the H-form, H-SSZ-39(N)-H-700.

### Example 4: Synthesis of SSZ-39(N)-H-800 Using Quaternary Ammonium Containing Structure Directing Agent and Calcination Thereof after Ammonium Ion Exchange at 800° C.

The method of Comparative Example 2 was repeated, wherein the ion exchanged product NH₄⁺-SSZ-39(N)-H was calcined in air in a muffle furnace at 800° C. for 5 hours which provided the H-form, H-SSZ-39(N)-H-800.

### Comparative Example 3: Synthesis of SSZ-39(P)-A Using Quaternary Phosphonium Containing Structure Directing Agent and Calcination Thereof in Air at 600° C.

The following synthesis of SSZ-39(P) is based on the synthetic methodology described in T. Sano et al., Chem. Lett. 2014, 43, page 302.

### Synthesis of Tetraethylphosphonium Hydroxide (TEPOH)

50 g of tetraethylphosphonium bromide (TCI, 98%) and 55 g of hydroxide ion exchange resin (DIAION SA10AOH, Mitsubishi Chemical) were mixed in distilled water. After 1 day, the resin was removed by filtration and the TEPOH aqueous solution with density of 1.075 g mL⁻¹ and molar concentration of 1.9 M was obtained.

### Synthesis of SSZ-39(P)

First, 5 g of TEPOH aqueous solution were mixed with 0.18 g of NaOH (Wako, 96%) and 0.45 g of distilled water. Then, 2.8 g of Y zeolite (CBV720, Si/Al₂=30, Zeolyst) were added to the above solution, with stirring for 1 h. The molar composition of the resultant gel was 1 SiO₂:0.067 Al:0.2 TEPOH:0.1 NaOH:5 H₂O. The thus prepared mother gel was crystallized in an autoclave at 170° C. for 5 days under tumbling condition (40 r.p.m.). The solid crystalline product, a zeolitic material having framework type AEI, was recovered by filtration, washed with distilled water, and dried overnight at 100° C. under air. The thus obtained product displayed an SiO₂: Al₂O₃ molar ratio of 24 as determined from elemental analysis by ICP.

The thus obtained SSZ-39(P) product was then calcined in air (A) in a muffle furnace at 600° C. for 6 hours which provided the sodium form, Na-SSZ-39(P)-A.

Subsequently, the Na-SSZ-39(P)-A was then NH₄⁺ ion exchanged using NH₄NO₃ in accordance with the treatment described in Comparative Example 1.

The thus obtained NH₄⁺-SSZ-39(P)-A was then calcined in air in a muffle furnace at 600° C. for 5 hours which provided the H-form, H-SSZ-39(P)-A-600.

### Example 5: Synthesis of SSZ-39(P)-A-700 Using Quaternary Phosphonium Containing Structure Directing Agent and Calcination Thereof after Ammonium Ion Exchange at 700° C.

The method of Comparative Example 3 was repeated, wherein the ion exchanged product NH₄⁺-SSZ-39(P)-A was calcined in air in a muffle furnace at 700° C. for 5 hours which provided the H-form, H-SSZ-39(P)-A-700.

### Example 6: Synthesis of SSZ-39(P)-A-800 Using Quaternary Phosphonium Containing Structure Directing Agent and Calcination Thereof after Ammonium Ion Exchange at 800° C.

The method of Comparative Example 3 was repeated, wherein the ion exchanged product NH₄⁺-SSZ-39(P)-A was calcined in air in a muffle furnace at 800° C. for 5 hours which provided the H-form, H-SSZ-39(P)-A-800.

### Comparative Example 4: Synthesis of SSZ-39(P)-H-600 Using Quaternary Phosphonium Containing Structure Directing Agent and Calcination Thereof in a Hydrogen-Atmosphere at 600° C.

The method of Comparative Example 3 was repeated, wherein the SSZ-39(P) product was calcined in a flow of hydrogen/nitrogen (H₂: 15 mL/min, N₂: 60 mL/min) (“H”) in a muffle furnace at 600° C. for 6 hours which provided the Na-SSZ-39(P)-H.

As in Comparative Example 1, the Na-SSZ-39(P)-H was then NH₄⁺ ion exchanged as described in Reference Example 1 to provide NH₄⁺-SSZ-39(P)-H, which was then calcined in air at 600° C. for 5 hours which provided the H-form, H-SSZ-39(P)-H-600.

### Example 7: Synthesis of SSZ-39(P)-H-700 Using Quaternary Phosphonium Containing Structure Directing Agent and Calcination Thereof after Ammonium Ion Exchange at 700° C.

The method of Comparative Example 2 was repeated, wherein the ion exchanged product NH₄⁺-SSZ-39(P)-H was calcined in air in a muffle furnace at 700° C. for 5 hours which provided the H-form, H-SSZ-39(P)-H-700.

### Example 8: Synthesis of SSZ-39(P)-H-800 Using Quaternary Phosphonium Containing Structure Directing Agent and Calcination Thereof after Ammonium Ion Exchange at 800° C.

The method of Comparative Example 2 was repeated, wherein the ion exchanged product NH₄⁺-SSZ-39(P)-H was calcined in air in a muffle furnace at 800° C. for 5 hours which provided the H-form, H-SSZ-39(P)-H-800.

### Example 9: Catalytic Testing in the Conversion of Methanol to Olefins (MTO)

The methanol-to-olefins (MTO) reaction was carried out at 350° C. under atmospheric pressure by using a fixed-bed reactor. Typically, 50 mg of 50/80 mesh zeolite pellets without a binder were loaded in a 6 mm quartz tubular flow microreactor and centered at the reactor in a furnace. The catalyst was activated in flowing He at 500° C. for 1 h prior to the reaction and then cooled to the desired reaction temperature. The pressure of methanol was set at 5 kPa. He was used as a carrier gas. W/F for methanol was set at 33.7 g-cat*h*mol⁻¹. The MTO reaction gives ethene (C2=), propene (C3=), butenes (C4=), paraffins (C1-C4), over-05 hydrocarbons, and dimethyl ether (DME) as products. The reaction products were analyzed by an online gas chromatograph (GC-2014, Shimadzu) equipped with an HP-PLOT/Q capillary column and an FID detector. The selectivities of the products were calculated on the basis of carbon number.

The results from the catalytic testing experiments for the examples and comparative examples are displayed in Tables 1 and 2 below.

TABLE 1
Results from methanol to olefin conversion testing performed with the
materials of Examples 1 to 4 and Comparative Examples 1 and 2.

| | conversion. | C2 = selectivity | C3 = selectivity | C4 = selectivity |
|---|---|---|---|---|
| Catalyst | >99% | initial (final) [%] | initial (final) [%] | initial (final) [%] |
| SSZ-39(N)-A-600 | 11 h | 21.3 (27.9) | 37.1 (43.2) | 14.3 (16.3) |
| (Comp. Example 1) | | | | |
| SSZ-39(N)-A-700 | 11 h | 21.2 (27.4) | 40.2 (45.6) | 15.2 (18.7) |
| (Example 1) | | | | |
| SSZ-39(N)-A-800 | 12 h | 20.3 (25.0) | 48.3 (46.8) | 19.2 (16.9) |
| (Example 2) | | | | |
| SSZ-39(N)-H-600 | 10 h | 21.6 (27.8) | 38.2 (42.9) | 15.1 (15.5) |
| (Comp. Example 2) | | | | |
| SSZ-39(N)-H-700 | 15 h | 18.6 (26.3) | 41.3 (45.9) | 17.6 (17.4) |
| (Example 3) | | | | |
| SSZ-39(N)-H-800 | 6 h | 19.7 (23.9) | 49.1 (47.3) | 18.8 (17.7) |
| (Example 4) | | | | |

As concerns the SSZ-39(N)-A catalysts wherein the organotemplate material was removed in air at 600° C., the results of which are displayed in Table 1, SSZ-39(N)-A-800 showed high C3= and C4=selectivities and long catalytic lifetime (12 h at >99% methanol conversion). Upon deactivation, the methanol conversion slowly decreased. This could be due to the decrease in the acid strength and amount when the catalyst was calcined at high temperature of 800° C.

Among SSZ-39(N)-H catalysts wherein the organotemplate material was removed in H₂/N₂ at 600° C., the results of which are displayed in Table 1, SSZ-39(N)-H-700 showed high C3= and C4=selectivities and long catalytic lifetime (15 h at >99% methanol conversion) compared to SSZ-39(N)-A-700. This could be due to the high amount of medium acids.

TABLE 2
Results from methanol to olefin conversion testing performed with the
materials of Examples 5 to 8 and Comparative Examples 3 and 4.

| | conversion. | C2 = selectivity | C3 = selectivity | C4 = selectivity |
|---|---|---|---|---|
| Catalyst | >99% | initial (final) [%] | initial (final) [%] | initial (final) [%] |
| SSZ-39(P)-A-600 | 7 h | 24.2 (33.3) | 46.3 (42.2) | 19.6 (15.9) |
| (Comp. Example 3) | | | | |
| SSZ-39(P)-A-700 | 4 h | 21.2 (23.3) | 48.1 (47.4) | 19.7 (18.7) |
| (Example 5) | | | | |
| SSZ-39(P)-A-800 | 1 h | 19.7 (20.6) | 48.3 (46.8) | 19.2 (20.0) |
| (Example 6) | | | | |
| SSZ-39(P)-H-600 | 15 h | 24.1 (33.7) | 41.1 (41.0) | 15.3 (14.2) |
| (Comp. Example 4) | | | | |
| SSZ-39(P)-H-700 | 7 h | 21.0 (27.8) | 46.5 (44.2) | 19.2 (16.8) |
| (Example 7) | | | | |
| SSZ-39(P)-H-800 | 4 h | 18.5 (24.6) | 47.3 (45.0) | 21.9 (17.9) |
| (Example 8) | | | | |

Regarding the SSZ-39(P)-A catalysts wherein the organotemplate material was removed in air at 600° C., the results of which are displayed in Table 2, these showed high C3= and C4=selectivities, however comparatively shorter catalytic lifetimes (1-7 h at >99% methanol conversion). This could be due to the low amount of acid sites with medium and strong acid strength.

Among SSZ-39(P)-H catalysts wherein the organotemplate material was removed in H₂/N₂ at 600° C., the results of which are displayed in Table 2, these showed long catalytic life time (up to 15 h at >99% methanol conversion), however comparatively lower C3= and C4=selectivities compared to SSZ-39(P)-H-700 or 800. This could be due to the high amount of medium and strong acid sites.

For investigating the influence of the acidic properties of the catalysts on their performance, NH₃-TPD measurements were performed on the fresh catalysts obtained according to the examples and comparative examples, the results of which are displayed in Tables 3 and 4 below.

TABLE 3
Deconvolution results from the NH₃-TPD measurements (temperature and
integration values of the deconvoluted desorption profile) performed on the materials of
Examples 1 to 4 and Comparative Examples 1 and 2.

| | peak I [mmol/g]/ | peak II [mmol/g]/ | peak III [mmol/g]/ |
|---|---|---|---|
| Catalyst | (temperature) | (temperature) | (temperature) |
| SSZ-39(N)-A-600 | 0.352/(390° C.) | 0.413/(480° C.) | 0.445/(173° C.) |
| (Comp. Example 1) | | | |
| SSZ-39(N)-A-700 | 0.196/(261° C.) | 0.427/(453° C.) | 0.366/(174° C.) |
| (Example 1) | | | |
| SSZ-39(N)-A-800 | 0.147/(217° C.) | 0.338/(340° C.) | 0.137/(168° C.) |
| (Example 2) | | | |
| SSZ-39(N)-H-600 | 0.406/(403° C. | 0.450/(483° C.) | 0.412/(172° C.) |
| (Comp. Example 2) | | | |
| SSZ-39(N)-H-700 | 0.218/(246° C.) | 0.380/(438° C.) | 0.280/(172° C.) |
| (Example 3) | | | |
| SSZ-39(N)-H-800 | 0.134/(217° C.) | 0.308/(335° C.) | 0.114/(169° C.) |
| (Example 4) | | | |

TABLE 4
Deconvolution results from the NH₃-TPD measurements (temperature and
integration values of the deconvoluted desorption profile) performed on the materials of
Examples 5 to 8 and Comparative Examples 3 and 4.

| | peak I [mmol/g]/ | peak II [mmol/g]/ | peak III [mmol/g]/ |
|---|---|---|---|
| Catalyst | (temperature) | (temperature) | (temperature) |
| SSZ-39(P)-A-600 | 0.249/(223° C.) | 0.329/(364° C.) | 0.201/(172° C.) |
| (Comp. Example 3) | | | |
| SSZ-39(P)-A-700 | 0.163/(209° C.) | 0.216/(321° C.) | 0.143/(164° C.) |
| (Example 5) | | | |
| SSZ-39(P)-A-800 | 0.143/(206° C.) | 0.153/(301° C.) | 0.114/(164° C.) |
| (Example 6) | | | |
| SSZ-39(P)-H-600 | 0.240/(251° C.) | 0.560/(442° C.) | 0.302/(172° C.) |
| (Comp. Example 4) | | | |
| SSZ-39(P)-H-700 | 0.174/(212° C.) | 0.310/(355° C.) | 0.160/(167° C.) |
| (Example 7) | | | |
| SSZ-39(P)-H-800 | 0.158/(207° C.) | 0.271/(328° C.) | 0.135/(165° C.) |
| (Example 8) | | | |

As may be taken from the results in Tables 3 and 4, it has surprisingly been found that the acid amount and strength of the SSZ-39(N) and SSZ-39(P) catalysts can be fine tuned by changing the calcination atmosphere and temperature, which allows for a substantial improvement in the C3= and C4=selectivities and catalytic lifetime as may be taken from the results from catalytic testing described in Tables 1 and 2. In particular, it has quite unexpectedly been found that the inventive zeolitic materials obtained according to the inventive method displaying specific quantities of acid sites and in particular displaying particular ratios of the amount of different acid sites to one another display both a considerably improved activity and a surprisingly high selectivity towards C2 to C4 olefins, and in particular towards C3 olefins in the catalytic conversion of methanol to olefins.

### LIST OF THE CITED PRIOR ART REFERENCES

Moliner, M. et al. in Chem. Commun. 2012, 48, pages 8264-8266
Maruo, T. et al. in Chem. Lett. 2014, 43, page 302-304
Martin, N. et al. in Chem. Commun. 2015, 51, 11030-11033
Unpublished international patent application PCT/CN2016/111314
Unpublished international patent application PCT/CN2017/112343
U.S. Pat. No. 5,958,370

1. A process for preparing a zeolitic material, the process comprising:
heating a mixture comprising a structure directing agent and a first zeolitic material comprising SiO₂ and X₂O₃ in its framework structure, the first zeolitic material having a FER, TON, MTT, FAU, GIS, MOR, BEA, MFI, and/or LTA framework structure,
to obtain a second zeolitic material having an AEI type framework structure comprising SiO₂ and X₂O₃ in its framework structure, X being a trivalent element;
optionally, first calcining the second zeolitic material;
optionally, subjecting the second zeolitic material to ion-exchange;
second calcining the second zeolitic material at a temperature in the range of from greater than 600 to 900° C.,
wherein the calcining of the second zeolitic material is effected under an atmosphere comprising less than 10 vol.-% of H₂O.

2. The process of claim 1, wherein the first and/or second calcining is effected under air as the atmosphere.

3. The process of claim 1, wherein the heating comprises heating the mixture at a temperature in a range of from 90 to 250° C.

4. The process of claim 1, wherein the heating is conducted under autogenous pressure.

5. The process of claim 1, wherein the first calcining is conducted and an atmosphere under which the first calcining is effected contains H₂ in a range of from 1 to 99 vol.

6. The process of claim 1, wherein X is Al, B, In, and/or Ga.

7. The process of claim 1, wherein the comprises OH⁻ source.

8. The process of claim 1, wherein the structure directing agent comprises a tetraalkylammonium cation compound comprising R¹R²R³R⁴N⁺,
wherein R¹, R², R³, and R⁴ are independently alkyl, and
wherein R³ and R⁴ form a common alkyl chain.

9. The process of claim 1, wherein the structure directing agent comprises a quaternary phosphonium cation compound comprising R¹R²R³R⁴P⁺,
wherein R¹, R², R³, and R⁴ are independently (C₁-C₆)alkyl.

10. A zeolitic material having an AEI typo framework structure, obtained by the process of claim 1.

11. A zeolitic material, having an AEI framework structure and comprising SiO₂ and X₂O₃ in its framework structure,
wherein X is a trivalent element,
wherein a deconvoluted ammonia temperature programmed desorption spectrum of the zeolitic material displays a first peak in a range of from 205 to 270° C. and a second peak in a range of from 300 to 460° C.,
wherein an integration of the first peak affords an amount of acid sites in a range of from 0.07 to 0.35 mmol/g, and
wherein an integration of the second peak affords an amount of acid sites in a range of from 0.25 to 0.4 mmol/g.

12. The zeolitic material of claim 11, wherein a ratio of an amount of acid sites from the integration of the first peak to an amount of acid sites from the integration of the second peak is in a range of from 0.35 to 0.7.

13. The zeolitic material of claim 11, having a CO-FTIR spectrum displaying a first peak in a range of from 3290 to 3315 cm⁻¹, and a second peak in a range of from 3420 to 3470 cm⁻¹,
wherein a maximum absorbance of the second peak is equal to or greater than a maximum absorbance of the first peak.

14. A process for converting one or more oxygenates to one or more olefins, the process comprising
contacting a gas stream, comprising an oxygenate.

15. A molecular sieve, catalyst, catalyst support, and/or as an adsorbent, comprising the zeolitic material of claim 11.

16. The process of claim 1, wherein X is Al.

17. The process of claim 1, wherein X is B.

18. The process of claim 1, wherein X is In.

19. The process of claim 1, wherein X is Ga.
